(19) United States
(12) Patent Application Publication
(10) Pub. No.: US 2025/0190863 A1
(43) Pub. Date: Jun. 12, 2025

(54) DRUG DELIVERY DEVICE WITH ELECTRONICS

(71) Applicant: Norton (Waterford) Limited, Waterford (IE)

(72) Inventors: Enrique Calderon Oliveras, Barcelona (ES); Anthony Kirwan, Waterford (IE); Eric Chan, Waterford (IE); Mark Milton-Edwards, Cheshire (GB); Michael Naumov, Beer Yakov (IL); Olga Khanikova, Waterford (IE)

(73) Assignee: Norton (Waterford) Limited, Waterford (IE)

(21) Appl. No.: 18/843,851

(22) PCT Filed: Apr. 12, 2023
(86) PCT No.: PCT/EP2023/059582

§ 371 Date: Sep. 4, 2024

Related U.S. Application Data
(60) Provisional application No. 63/330,118, filed on Apr. 12, 2022.

(51) Int. Cl. G06N 20/00; G06K 7/14

(52) U.S. Cl. CPC G06N 20/00; G06K 7/1417

(57) ABSTRACT

A system may be configured for sending a Uniform Resource Locator (URL) request based on a location of an external device (502). An inhaler (100) may include medicament, an electronics module (120), and/or a Quick Response (QR) code (160). An external device may determine a URL and/or a medicament type of the inhaler (100) based on the QR code (160). The external device may send an indication of the medicament type and/or a location indication to a server that hosts the URL. In response, the external device (502) may receive an application store URL that is specific to the medicament type and/or the location of the external device. The external device may send a request to the application store URL to download software that is specific to the inhaler (100) and/or the medicament type of the inhaler. The application store URL may be specific to a country that the external device is located.

### CROSS-REFERENCE TO RELATED APPLICATIONS

This application claims the benefit of Provisional U.S. Patent Application No. 63/330,118, filed Apr. 12, 2022, the disclosure of which is incorporated herein by reference in its entirety.

### BACKGROUND

Drug delivery devices facilitate the delivery of medication into a patient's body via various routes of administration. Typical routes of administration include oral, topical, sublingual inhalation, injection and the like. The devices may be used to deliver medications for the treatment of various diseases, ailments and medical conditions. Inhalation devices, for example, may be used to treat asthma, chronic obstructive pulmonary disease (COPD) and cystic fibrosis (CF). While drug delivery devices may be designed to deliver an appropriate dose of medication to a patient as part of a therapeutic treatment, the effectiveness of the treatment may be influenced by non-physiological factors, such as the patient's adherence and compliance in using the device.

Drug delivery devices may be equipped with sensors to track adherence and compliance. For example, a device may include a sensor for detecting when the device was actuated to deliver a dose of medication and/or its orientation during actuation. This information may be stored in a local memory and subsequently communicated to another device, such as a smartphone, tablet or computer, for further processing. The device's actuation history may be compared to a dosing regimen prescribed by a physician to determine the patient's level of adherence. Other sensor data, such as the device's orientation during operation, may be reviewed to determine whether the patient is using the device in a compliant manner that facilitates proper delivery of the medication.

To support the addition of sensors and communication mechanisms, a drug delivery device may include a local power source. If the drug delivery device is portable, the local power source may be portable as well. For example, the local power source may be a small battery. Certain factors, such as size, weight, and/or cost, may limit the types of viable power sources for the device. Accordingly, a finite amount of power may be available when choosing a power source for a drug delivery device with electrical components.

### SUMMARY

A drug delivery device may be adapted to include an electronics module that is configured to sense, track, and/or process usage conditions and parameters associated with the device (e.g., to improve adherence and compliance). The electronics module may be further configured to communicate the conditions and parameters to external devices, such as a smartphone, for similar and/or further processing. The inclusion of an electronics module in a drug delivery device opens the doors to a wealth of digital improvements and features to enhance the use of the device. The electronics module, in this context, may create a platform to leverage helpful smartphone applications and powerful data analytics. However, the introduction of electronics into any drug delivery device may introduce certain technical challenges, such as durability, reliability, electro-mechanical integration, power management, and drug delivery performance. The present disclosure provides solutions for inclusion of certain electrical components with a drug delivery device, such as an inhaler.

Examples of inhalation devices (e.g., breath-actuated inhalers) are provided herein. A system may be configured for sending a Uniform Resource Locator (URL) request based on a location of an external device. The system may comprise an inhaler, an external device, and/or a server. The inhaler may include medicament, an electronics module, and/or a machine readable code—for example, a Quick Response (QR) code or other barcode. The electronics module may include a processor, memory, and/or a communication circuit. The external device may be configured to determine a URL based on the machine readable code. The external device may be configured to determine a medicament type of the inhaler based on the machine readable code. The external device may be configured to send an indication of the medicament type and/or a location indication to a server that hosts the URL. In response to sending the indication of the medicament type and/or the location indication, the external device may be configured to receive an application store URL that is specific to the medicament type and/or the location of the external device. The external device may be configured to send a request to the application store URL to download software that is specific to the inhaler and/or the medicament type of the inhaler. The application store URL may be specific to the country that the external device is located. By “specific to the inhaler” it is meant that the software is specially adapted to interact with inhalers of a certain predetermined class—for example, inhalers having a certain kind code, product code, or model number. It does not mean that the software is unique to a single individual inhaler.

The external device may be configured to determine a location of the external device. The external device may send an indication of the location of the external device to the server that hosts the URL. In response, the external device may receive the application store URL that is specific to the medicament type and/or the location of the external device. The external device may be configured to determine a product identifier (ID) of the inhaler based on the image of the machine readable code. The external device may determine the medicament type based on the product ID. The product ID may comprise a multi-digit alphanumeric code that indicates the medicament type of the inhaler. The external device may be configured to determine a communication passkey that is unique to the inhaler based on the machine readable code. The external device may be configured to transmit the communication passkey to the electronics module of the inhaler, for example, to enable communication between the electronics module and the external device. The communication passkey may include a Bluetooth Low Energy (BLE) passkey. The external device may be configured to determine the location indication based on a preconfigured setting of the external device. The external device may be configured to receive, in response to sending the indication of the medicament type and the location indication, a URL associated with inhaler support. The external device may be configured to receive the URL associated with inhaler support when a location of the external device is not within a predetermined geographic region.

The external device may be configured to receive software that is specific to the inhaler and the medicament type of the inhaler. The external device may be configured to install an application using the software that is specific to the inhaler and the medicament type of the inhaler.

A system comprising an inhaler, an external device, and one or more servers may be configured to redirect a URL request to an application store URL. The inhaler may include medicament, an electronics module, and/or a machine readable code—for example, a Quick Response (QR) code or other barcode. The electronics module may include a processor, memory, and a communication circuit. The external device may be configured to determine a URL based on the machine readable code and/or a medicament type of the inhaler based on the machine readable code. The external device may be configured to send an indication of the medicament type and a location indication to a server that hosts the URL. The server may be configured to receive the indication of the medicament type and a location indication at the URL. The server may be configured to determine that the medicament type is associated with a mobile application that is specific to the inhaler and/or the medicament type. The server may be configured to determine whether the location indication matches a predetermined value. The server may be configured to redirect the external device to an application store URL that is specific to the inhaler and the medicament type based upon a determination that the location indication matches the predetermined value. The external device may be configured to receive the application store URL that is specific to the medicament type. The external device may be configured to send a request to the application store URL to download software that is specific to the inhaler and the medicament type of the inhaler.

A system comprising an inhaler, an external device, and/or one or more servers may be configured to determine whether to include inhaler data from a specific inhaler in an analysis associated with a user of the inhaler based on whether the specific inhaler is a demonstrator inhaler. A demonstrator inhaler may be an inhaler that does not comprise medicament. The external device may be configured to receive an image of a machine readable code (for example, a QR code or other barcode) located on the inhaler. The external device may be configured to determine, based on the image of the machine readable code, that the inhaler is a demonstrator inhaler. The external device may be configured to send, to a remote server, an indication that the inhaler is a demonstrator inhaler. The remote server may be configured to receive inhaler data associated with the use of the inhaler and the indication that the inhaler is a demonstrator inhaler. The inhaler data may include one or more of an event record, one or more inhalation parameters, one or more environmental conditions, one or more usage parameters, or actuation of an internal switch of the inhaler. The remote server may be configured to determine, based on the indication that the inhaler is a demonstrator inhaler, to not use the inhaler data associated with the use of the inhaler in an analysis associated with a user of the inhaler. The analysis associated with the user may include an artificial intelligence model. Additionally or alternatively, the analysis associated with the user may include a predictive analysis associated with exacerbation—for example, to determine a predicted likelihood (probability) of a respiratory exacerbation.

The inhaler may include a sensor configured to detect the one or more inhalation parameters associated with a use of the inhaler by a user. The electronics module of the inhaler may be configured to send the one or more inhalation parameters to the external device. The external device may be configured to receive the one or more inhalation parameters from the inhaler, for example, the electronics module. The external device may be configured to send, to the remote server, the one or more inhalation parameters along with the indication that the inhaler is a demonstrator inhaler. The external device may be configured to determine a location of the external device based on a preconfigured setting of the external device. The external device may be configured to send an indication of the location to the remote server.

A system comprising one or more inhalers, one or more external devices, and one or more servers may be configured to determine whether to use data from an inhaler in a predictive analysis associated with a user of the inhaler. A first external device may receive an image of a machine readable code (for example, a QR code or other barcode) of a first inhaler. The first external device may determine, based on the image of the machine readable code of the first inhaler, that the first inhaler is a demonstrator inhaler. The first external device may send, to a remote server, the first inhalation parameter and an indication that the first inhaler is a demonstrator inhaler.

A second external device may receive an image of a machine readable code of a second inhaler. The second external device may determine, based on the image of the machine readable code of the second inhaler, that the second inhaler comprises medicament and is not a demonstrator inhaler. The second external device may send, to the remote server, the second inhalation parameter. The remote server may receive the first inhalation parameter associated with the first inhaler and the indication that the first inhaler is a demonstrator inhaler. The remote server may receive the second inhalation parameter associated with the second inhale. The remote server may determine, based on the indication that the first inhaler is a demonstrator inhaler, to use the second inhalation parameter but not the first inhalation parameter in an artificial intelligence model.

An inhaler may include a mouthpiece and a mouthpiece cover. The mouthpiece cover may be configured to cover the mouthpiece. The inhaler may include an electronics module that includes a processor, memory, and a communication circuit. The inhaler may include a machine readable code (for example, a QR code or other barcode) having an indication that the inhaler does not comprise medicament. The machine readable code may include an indication of a URL associated with the inhaler. The URL may include an application store URL associated with an inhaler application. The indication may indicate that the inhaler is a demonstrator inhaler when the inhaler does not comprise medicament.

A method for training a predictive model may be provided that is configured to identify demonstrator inhalers that do not comprise medicament. The method may include receiving a plurality of first event records generated by a plurality of first inhalers (for example, inhalers containing a medicament). An event record may be generated by an inhaler in response to an inhaler event. The inhaler event may include actuation of a switch and/or receipt of measurements from a sensor of the inhaler. Each of the plurality of first event records may be associated with a single inhaler of the plurality of first inhalers, associated with a day and a time of a respective one of the plurality of first event records, associated with a respective user of a plurality of first users, and/or associated with a flow rate. The method may include receiving a plurality of second event records generated by a plurality of second inhalers. The plurality of second inhalers may be demonstrator inhalers that do not comprise medicament. Each of the plurality of second event records may be associated with a single inhaler of the plurality of second inhalers, a day and a time of a respective one of the plurality of second event records, a respective user of a plurality of second users, and/or a flow rate. The method may include training a predictive model, using the plurality of first and second event records, to identify one or more parameters that are associated with demonstrator inhalers. The method may include receiving a third event record generated by a third inhaler. The method may further include determining, using the trained predictive model, whether the third inhaler is a demonstrator inhaler that does not comprise medicament.

The predictive model may be trained to determine that the third inhaler is a demonstrator inhaler based on inhalation events of the third inhaler not occurring between a daily time period associated with nighttime. The predictive model may be trained to determine that the third inhaler is a demonstrator inhaler based on inhalation events of the third inhaler not occurring on Saturday or Sunday. The predictive model may be trained to determine that the third inhaler is a demonstrator inhaler based on a user associated with the third inhaler having an age that is above a lower threshold or below an upper threshold. The predictive model may be trained to determine that third inhaler is a demonstrator inhaler based on the third inhaler being associated with a number of inhalation events that exceeds a threshold (e.g., 200 uses) within a predetermined time period. The predictive model may be trained to determine that the third inhaler is a demonstrator inhaler based on the third inhaler having a dose count that exceeds an expected number of doses of medicament (e.g., greater than 70 doses in an inhaler that traditionally only has 60 doses). The predictive model may be trained to determine that the third inhaler is a demonstrator inhaler based on a percentage of the flow rates of a plurality of event records associated with the third inhaler being above a threshold flow rate. The predictive model may be trained to determine that the third inhaler is not a demonstrator inhaler based on the inverse of any of the criteria mentioned above (for example, based on the third inhaler having a dose count that does not exceed an expected number of doses of medicament), or based on a combination of any two or more such inverse criteria. The predictive model may be trained using an unsupervised learning method (e.g., a clustering method, such as a k-means or c-means clustering method). The predictive model may be trained using a supervised learning method (e.g., gradient boosted decision trees and/or an XGBoost algorithm).

The method may include training a second predictive model, using the plurality of first event records from the plurality of first inhalers and determining, using the second predictive model, a likelihood of a respiratory exacerbation of a respective user of the plurality of first users. The method may further include receiving a fourth inhaler record from a fourth inhaler and determining, using the second predictive model, a likelihood of a respiratory exacerbation of a user associated with the fourth inhaler.

### BRIEF DESCRIPTION OF THE DRAWINGS

FIG. 1 is a front perspective view of an example inhalation device.

FIG. 2 is a cross-sectional interior perspective view of the example inhalation device.

FIG. 3 is an exploded perspective view of the inhalation device with a top cap removed to expose an electronics module.

FIG. 4 is an exploded perspective view of the top cap and the electronics module of the inhalation device.

FIG. 5 is a diagram of an example system including a plurality of inhalation devices.

FIG. 6 is a flow diagram that illustrates an example process for sending a Uniform Resource Locator (URL) request based on a location of an external device.

FIG. 7 is a flow diagram that illustrates an example process for identifying a URL to visit based on a Quick Response (QR) code.

FIG. 8 is a flow diagram that illustrates an example process for excluding data from a demonstrator inhaler from one or more analyses.

FIG. 9 is a flow diagram that illustrates an example process for training a model to determine whether an inhaler is a demonstrator inhaler.

FIG. 10 is a graph of exemplary airflow rates through the example inhalation device of FIG. 1 based on pressure measurements recorded by the electronics module.

FIG. 11 is a block diagram of an example electronics module of the example inhalation device of FIG. 1.

FIG. 12 is a block diagram of an example external device.

### DETAILED DESCRIPTION

The present disclosure describes devices, systems and methods for sensing, tracking and/or processing usage conditions and parameters associated with a drug delivery device. The devices, systems and methods are described in the context of a breath-actuated inhalation device for delivering medication into a user's lungs. However, the described solutions are equally applicable to other drug delivery devices, such as an injector, a metered-dose inhaler, a nebulizer, a transdermal patch, or an implantable.

Asthma and COPD are chronic inflammatory disease of the airways. They are both characterized by variable and recurring symptoms of airflow obstruction and bronchospasm. The symptoms include episodes of wheezing, coughing, chest tightness and shortness of breath. The symptoms are managed by avoiding triggers and by the use of medicaments, particularly inhaled medicaments. The medicaments include inhaled corticosteroids (ICSs) and bronchodilators.

Inhaled corticosteroids (ICSs) are steroid hormones used in the long-term control of respiratory disorders. They function by reducing the airway inflammation. Examples include budesonide, beclomethasone (dipropionate/dipropionate HFA), fluticasone (propionate), mometasone (furoate), ciclesonide and dexamethasone (sodium). Parentheses indicate examples (e.g., preferred salt or ester forms).

Different classes of bronchodilators target different receptors in the airways. Two commonly used classes are β2-agonists and anticholinergics. β2-Adrenergic agonists (or “β2-agonists”) act upon the β2-adrenoceptors which induces smooth muscle relaxation, resulting in dilation of the bronchial passages. They tend to be categorised by duration of action. Examples of long-acting β2-agonists (LABAs) include formoterol (fumarate), salmeterol (xinafoate), indacaterol (maleate), bambuterol (hydrochloride), clenbuterol (hydrochloride), olodaterol (hydrochloride), carmoterol (hydrochloride), tulobuterol (hydrochloride) and vilanterol (triphenylacetate). Examples of short-acting β2-agonists (SABA) are albuterol (sulfate) and terbutaline (sulfate).

Typically, short-acting bronchodilators provide a rapid relief from acute bronchoconstriction (and are often called “rescue” or “reliever” medicines), whereas long-acting bronchodilators help control and prevent longer-term symptoms. However, some rapid-onset long-acting bronchodilators may be used as rescue medicines, such as formoterol (fumarate). Thus, a rescue medicine provides relief from acute bronchoconstriction. The rescue medicine is taken as-needed/prn (pro re nata). The rescue medicine may also be in the form of a combination product, e.g., ICS-formoterol (fumarate), typically budesonide-formoterol (fumarate) or beclomethasone (dipropionate)-formoterol (fumarate). Thus, the rescue medicine is preferably a SABA or a rapid-acting LABA, more preferably albuterol (sulfate) or formoterol (fumarate), and most preferably albuterol (sulfate).

Anticholinergics (or “antimuscarinics”) block the neurotransmitter acetylcholine by selectively blocking its receptor in nerve cells. On topical application, anticholinergics act predominantly on the M3 muscarinic receptors located in the airways to produce smooth muscle relaxation, thus producing a bronchodilatory effect. Examples of long-acting muscarinic antagonists (LAMAs) include tiotropium (bromide), oxitropium (bromide), aclidinium (bromide), umeclidinium (bromide), ipratropium (bromide) glycopyrronium (bromide), oxybutynin (hydrochloride or hydrobromide), tolterodine (tartrate), trospium (chloride), solifenacin (succinate), fesoterodine (fumarate) and darifenacin (hydrobromide).

A number of approaches have been taken in preparing and formulating these medicaments for delivery by inhalation, such as via a dry powder inhaler (DPI), a pressurized metered dose inhaler (pMDI) or a nebulizer.

According to the GINA (Global Initiative for Asthma) Guidelines, a step-wise approach can be taken to the treatment of asthma. At step 1, which represents a mild form of asthma, the patient is given an as needed SABA, such as albuterol sulfate. The patient may also be given an as-needed low-dose ICS-formoterol, or a low-dose ICS whenever the SABA is taken. At step 2, a regular low-dose ICS is given alongside the SABA, or an as-needed low-dose ICS-formoterol. At step 3, a LABA is added. At step 4, the doses are increased and at step 5, further add-on treatments are included such as an anticholinergic or a low-dose oral corticosteroid. Thus, the respective steps may be regarded as treatment regimens, which regimens are each configured according to the degree of acute severity of the respiratory disease.

COPD is a leading cause of death worldwide. It is a heterogeneous long-term disease comprising chronic bronchitis, emphysema and also involving the small airways. The pathological changes occurring in patients with COPD are predominantly localized to the airways, lung parenchyma and pulmonary vasculature. Phenotypically, these changes reduce the healthy ability of the lungs to absorb and expel gases.

Bronchitis is characterized by long-term inflammation of the bronchi. Common symptoms may include wheezing, shortness of breath, cough and expectoration of sputum, all of which are highly uncomfortable and detrimental to the patient's quality of life. Emphysema is also related to long-term bronchial inflammation, wherein the inflammatory response results in a breakdown of lung tissue and progressive narrowing of the airways. In time, the lung tissue loses its natural elasticity and becomes enlarged. As such, the efficacy with which gases are exchanged is reduced and respired air is often trapped within the lung. This results in localised hypoxia, and reduces the volume of oxygen being delivered into the patient's bloodstream, per inhalation. Patients therefore experience shortness of breath and instances of breathing difficulty.

Patients living with COPD experience a variety, if not all, of these symptoms on a daily basis. Their severity will be determined by a range of factors but most commonly will be correlated to the progression of the disease. These symptoms, independent of their severity, are indicative of stable COPD and this disease state is maintained and managed through the administration of a variety drugs. The treatments are variable, but often include inhaled bronchodilators, anticholinergic agents, long-acting and short-acting β2-agonists and corticosteroids. The medicaments are often administered as a single therapy or as combination treatments.

Patients are categorized by the severity of their COPD using categories defined in the GOLD Guidelines (Global Initiative for Chronic Obstructive Lung Disease, Inc.). The categories are labelled A-D and the recommended first choice of treatment varies by category. Patient group A are recommended a short-acting muscarinic antagonist (SAMA) prn or a short-acting β2-aginist (SABA) prn. Patient group B are recommended a long-acting muscarinic antagonist (LAMA) or a long-acting β2-aginist (LABA). Patient group C are recommended an inhaled corticosteroid (ICS)+a LABA, or a LAMA. Patient group D are recommended an ICS+a LABA and/or a LAMA.

Patients suffering from respiratory diseases like asthma or COPD suffer from periodic exacerbations beyond the baseline day-to-day variations in their condition. An exacerbation is an acute worsening of respiratory symptoms that require additional therapy, i.e., a therapy going beyond their maintenance therapy.

For asthma, the additional therapy for a moderate exacerbation are repeated doses of SABA, oral corticosteroids and/or controlled flow oxygen (the latter of which requires hospitalization). A severe exacerbation adds an anticholinergic (typically ipratropium bromide), nebulized SABA or IV magnesium sulfate.

For COPD, the additional therapy for a moderate exacerbation are repeated doses of SABA, oral corticosteroids and/or antibiotics. A severe exacerbation adds controlled flow oxygen and/or respiratory support (both of which require hospitalization). An exacerbation within the meaning of the present disclosure includes both moderate and severe exacerbations.

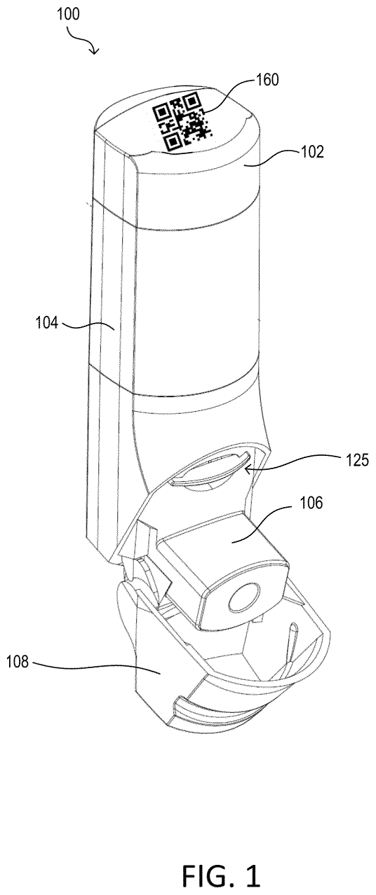

FIG. 1 is a front perspective view of an example inhalation device 100. The example, inhalation device 100 may be a breath-actuated inhalation device. The inhalation device 100 may include a top cap 102, a main housing 104, a mouthpiece 106, a mouthpiece cover 108, medicament, and an air vent 125. The top cap 102 may be mechanically attached to the main housing 104. The mouthpiece cover 108 may be hinged to the main housing 104 so that it may open and close to expose the mouthpiece 106. Although illustrated as a hinged connection, the mouthpiece cover 108 may be connected to the inhalation device 100 through other types of connections.

The inhalation device 100 may include a rescue medicament or a maintenance medicament. The rescue medicament may be a SABA or a rapid-onset LABA, such as formoterol (fumarate). The rescue medicament may also be in the form of a combination product, e.g., ICS-formoterol (fumarate), typically budesonide-formoterol (fumarate) or beclomethasone (dipropionate)-formoterol (fumarate). Such an approach is termed “MART” (maintenance and rescue therapy). In some examples, the medicament is albuterol (sulfate), fluticasone (propionate or furoate), or salmeterol (xinafoate) combined with fluticasone (propionate or furoate).

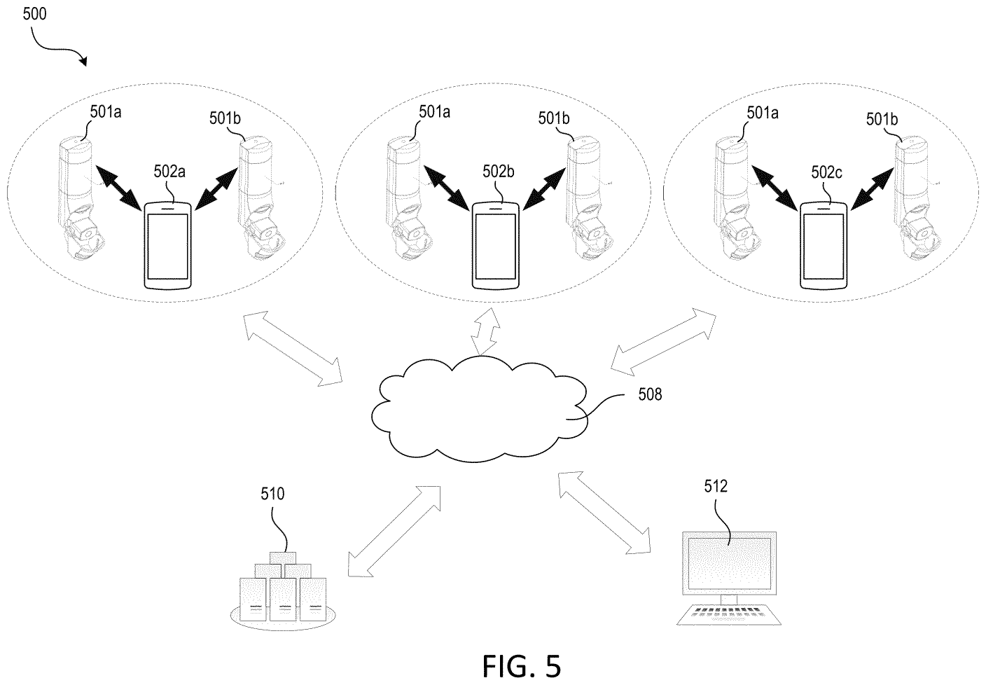

The inhalation device 100 may include a barcode, such as a Quick Response (QR) code 160 that is used to facilitate the pairing process between the inhalation device 100 and a mobile device (e.g., such as one of the mobile devices 502a, 502b, 502c shown in FIG. 5). For instance, in some examples, the inhalation device 100 does not include an actuator, button, or switch to initiate a pairing process with a mobile device, and as such, the QR code 160 may be used. Although described as a QR code 160, other types of barcodes may be used. The use of the QR code 160 to initiate the pairing process may further reduce the required battery/power consumption of the electronics module of the inhalation device 100. Further, although the QR code 160 is illustrated as being located on the top of the top cap 102, in other examples, the inhalation device 100 may include a QR code 160 that is located elsewhere on the inhalation device 100, such as on the main housing 104 or on the mouthpiece cover 108. The mobile device may include a camera, and the mobile device may be configured to access the camera and read the QR code 160.

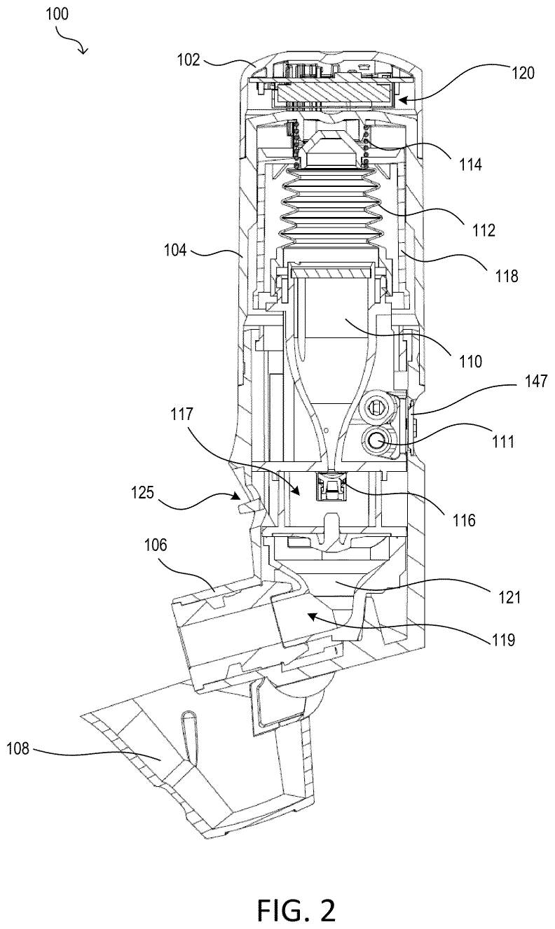

FIG. 2 is a cross-sectional interior perspective view of the inhalation device 100. Inside the main housing 104, the inhalation device 100 may include a medication reservoir and a dose delivery mechanism/system. For example, the inhalation device may include a medication reservoir 110 (e.g., a hopper), a bellows 112, a bellows spring 114, a yoke 118, a dose counter 111, a transparent window 147, a dosing cup 116, a dosing chamber 117, a deagglomerator 121 and a flow pathway 119. The medication reservoir 110 may include medication, such as dry powder mediation, which may be delivered to the user via the mouthpiece 106. The yoke 118 may be mechanically coupled (e.g., directly or indirectly) with the mouthpiece cover 108 such that a movement of the mouthpiece cover 108 may result in a movement of the yoke 118. For example, when the mouthpiece cover 108 is moved to expose the mouthpiece 106 (e.g., from a closed position to an open position), the yoke 118 may move vertically (e.g., towards or away from the top cap 102) within the inhalation device 100.

The movement of the yoke 118 may cause the bellows 112 to compress, thereby delivering a dose of medication from the medication reservoir 110 to the dosing cup 116. Thereafter, a user may inhale through the mouthpiece 106 to receive the dose of medication. The airflow generated from the user's inhalation may cause the deagglomerator 121 to aerosolize the dose of medication by breaking down the agglomerates of the medication in the dose cup 116. The deagglomerator 121 may be configured to (e.g., fully) aerosolize the medication when the airflow through the flow pathway 119 meets or exceeds a rate or is within a specific range. When aerosolized, the dose of medication may travel from the dosing cup 116, into the dosing chamber 117, through the flow pathway 119, and out of the mouthpiece 106 to the user. If the airflow through the flow pathway 119 does not meet or exceed a rate, or is not within a specific range, all or a portion of the medication may remain in the dosing cup 116. In the event that the medication in the dosing cup 116 has not been aerosolized by the deagglomerator 121, another dose of medication may not be delivered from the medication reservoir 110 when the mouthpiece cover 108 is subsequently opened. Thus, at least a portion of a dose of medication may remain in the dosing cup 116 until the dose has been aerosolized by the deagglomerator 121.

As the user inhales through the mouthpiece 106, air may enter the air vent 125 to provide a flow of air for delivery of the medication to the user. The flow pathway 119 may extend from the dosing chamber 117 to the end of the mouthpiece 106. The flow pathway may include the dosing chamber 117 and the internal portions of the mouthpiece 106. The dosing cup 116 may reside within or adjacent to the dosing chamber 117.

Although illustrated as a combination of the bellows 112, the bellows spring 114, the yoke 118, the dosing cup 116, the dosing chamber 117, and the deagglomerator 121, the dose delivery mechanism may include a subset of the components described and/or the inhalation device 100 may include a different dose delivery mechanism (e.g., based on the type of inhalation device, the type of medication, etc.). For instance, in some examples the medication may be included in a blister strip and the dose delivery mechanism (e.g., one or more wheels, levers, and/or actuators) may be configured to advance the blister strip, open a new blister that includes a dose of medication, and make that dose of medication available to a dosing chamber and/or mouthpiece for inhalation by the user.

In the illustrated example dose delivery mechanism of FIG. 1, the dose counter 111 may be mechanically coupled (e.g., directly or indirectly) with the mouthpiece cover 108 such that the dose counter 111 may increment or decrement when the mouthpiece cover 108 is opened or closed. The dose counter 111 may initially be set to a number of total doses available, which may be the number of doses in the medication reservoir 110 or the number of doses advertised by the manufacturer. As such, the dose counter 111 may be configured to decrease by one each time the mouthpiece cover 108 is moved from the closed position to the open position (or from the open position to the closed position), thereby indicating the remaining number of doses available. Alternatively, the dose counter 111 may initially be set to zero and may be configured to increase by one each time the mouthpiece cover 108 is moved from the closed position to the open position (or from the open position to the closed position), thereby indicating the total number of doses delivered from the medication reservoir 110.

The inhalation device 100 may include an electronics module 120, which may be housed within the top cap 102. The electronics module 120 may include a printed circuit board (PCB) assembly 122 with one or more electrical components, such as a sensor system 128 and a wireless communication circuit 129. The sensor system 128 may be configured to detect one or more parameters associated with the usage of the device and/or the environment in which the device is used or stored. The wireless communication circuit 129 may be configured to transmit the detected parameters to an external device, such as a smartphone, tablet, or computer, for further processing.

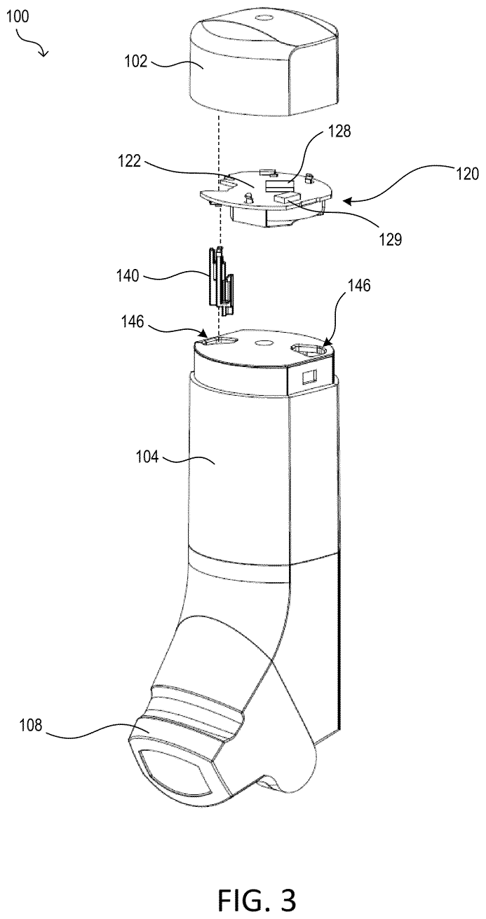

FIG. 3 is an exploded perspective view of the example inhalation device 100 with the top cap 102 removed to expose the electronics module 120. The top cap 102 may house the electronics module 120, which may include a printed circuit board (PCB) assembly 122. The PCB assembly 122 may include one or more components, such as a sensor system 128 and a wireless communication circuit 129. The top cap 102 may be attached to the main housing 104 via one or more clips (not shown) that engage recesses on the main housing 104. The top cap 102 may overlap a portion of the main housing 104 when connected, for example, such that a substantially pneumatic seal exists between the top cap 102 and the main housing 104. The top surface of the main housing 104 may include one or more (e.g., two) orifices 146. One of the orifices 146 may be configured to accept a slider 140. For example, when the top cap 102 is attached to the main housing 104, the slider 140 may protrude through the top surface of the main housing 104 via one of the orifices 146. The top cap 102 may be removably attached to the main housing 104. Alternatively or additionally, the electronics module 120 may be integrated within the main housing 104 and/or the top cap 102 housing the electronics module 120 may be permanently attached to the main housing 104.

Further, in some examples, the electronics module 120 may reside is a separate device that is outside of and separate from the inhalation device 100. For instance, the electronics module 120 may reside within an add-on device that is configured to be attached to and subsequently removed from the inhalation device 100, for example, when the inhalation device 100 runs out of medication or expires. In such instances, the user may attach the add-on device that includes the electronics module 120 from one inhalation device 100 to another each time the user receives a new inhalation device 100. The add-on device may be configured to be attached to any component of the inhalation device 100, such as the main housing 104, the mouthpiece, and/or a medication canister housed within the main housing of the main housing 104 of the inhalation device 100 (e.g., such that the sensors are in fluid communication with the mouthpiece and/or flow channel of inhalation device 100. As such, in some examples, the inhalation device 100 may be replaced by an add-on device that includes the electronics module 120 (e.g. in whole or in part), and possibly an inhaler that does not include electronics.

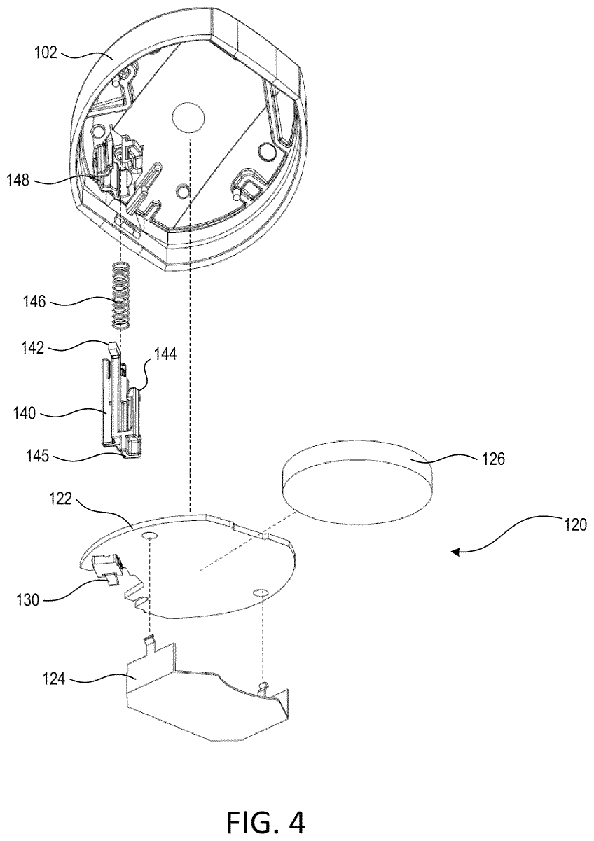

FIG. 4 is an exploded perspective view of the top cap 102 and the electronics module 120. As shown in FIG. 4, the slider 140 may define an arm 142, a stopper 144, and a distal base 145. The distal end 145 may be a bottom portion of the slider 140. The distal end 145 of the slider 140 may be configured to abut the yoke 118 that resides within the main housing 104. The top cap 102 may include a slider guide 148 that is configured to receive a slider spring 146 and the slider 140. The slider spring 146 may reside within the slider guide 148. The slider spring 146 may engage an inner surface of the top cap 102, and the slider spring 146 may engage (e.g., abut) an upper portion (e.g., a proximate end) of the slider 140. When the slider 140 is installed within the slider guide 148, the slider spring 146 may be partially compressed between the top of the slider 140 and the inner surface of the top cap 102. For example, the slider spring 146 may be configured such that the distal end 145 of the slider 140 remains in contact with the yoke 118 when the mouthpiece cover 108 is closed.

The distal end 145 of the slider 145 may also remain in contact with the yoke 118 while the mouthpiece cover 108 is opened or closed. The stopper 144 of the slider 140 may engage a stopper of the slider guide 148, for example, such that the slider 140 is retained within the slider guide 148 through the opening and closing of the mouthpiece cover 108, and vice versa. The stopper 144 and the slider guide 148 may be configured to limit the vertical (e.g., axial) travel of the slider 140. This limit may be less than the vertical travel of the yoke 118. Thus, as the mouthpiece cover 108 is moved to an open position, the yoke 118 may continue to move in a vertical direction towards the mouthpiece 106 but the stopper 144 may stop the vertical travel of the slider 140 such that the distal end 145 of the slider 140 may no longer be in contact with the yoke 118.

As noted above, the electronics module 120 may include one or more components, such as the sensor system 128 and the wireless communication circuit 129. The electronics module 120 may further include a switch 130, a power supply 126 (e.g., a battery), a power supply holder 124, an indicator (e.g., a light emitting diode (LED)), a controller (e.g., processor) and/or memory. When used herein, the terms controller and processor may be used interchangeably. One or more of the components of the electronics module 120 may be mounted on, and electrically coupled to, the PCB 122. The controller and/or memory may be physically distinct components of the PCB 122. Alternatively, the controller and memory may be part of a chipset mounted on the PCB 122. For example, the wireless communication circuit 129 may include the controller and/or memory for the electronics module 120. The controller of the electronics module 120 may include a microcontroller, a programmable logic device (PLD), a microprocessor, an application specific integrated circuit (ASIC), a field-programmable gate array (FPGA), or any suitable processing device or control circuit. The memory may include computer-executable instructions that, when executed by the controller, cause the controller to implement the processes of the electronics module as described herein.

The controller may access information from, and store data in the memory. The memory may include any type of suitable memory, such as non-removable memory and/or removable memory. The non-removable memory may include random-access memory (RAM), read-only memory (ROM), a hard disk, or any other type of memory storage device. The removable memory may include a subscriber identity module (SIM) card, a memory stick, a secure digital (SD) memory card, and the like. The memory may be internal to the controller. The controller may also access data from, and store data in, memory that is not physically located within the electronics module 120, such as on a server or a smartphone.

The memory may comprise a computer-readable storage media or machine-readable storage media that maintains computer-executable instructions for performing one or more as described herein. For example, the memory may comprise computer-executable instructions or machine-readable instructions that include one or more portions of the procedures described herein. The controller of the electronics module 120 may access the instructions from memory for being executed to cause the controller of the electronics module 120 to operate as described herein, or to operate one or more other devices as described herein. The memory may comprise computer-executable instructions for executing configuration software. For example, the computer-executable instructions may be executed to perform, in part and/or in their entirety, one or more procedures, such as the procedures 600, 700, 800, and/or 900 as described herein. Further, the memory may have stored thereon one or more settings and/or control parameters associated with the electronics module 120.

The battery 126 may provide power to the components of the PCB 122. The battery 126 may be any suitable source for powering the electronics module 120, such as a coin cell battery, for example. The battery 126 may be rechargeable or non-rechargeable. The battery 126 may be housed by the battery holder 124. The battery holder 124 may be secured to the PCB 122 such that the battery 126 maintains continuous contact with the PCB 122 and/or is in electrical connection with the components of the PCB 122. The battery 126 may have a battery capacity that may affect the life of the battery 126. As will be further discussed below, the distribution of power from the battery 126 to the one or more components of the PCB 122 may be managed to ensure the battery 126 can power the electronics module 120 over the useful life of the inhalation device 100 and/or the medication contained therein.

The switch 130 may be actuated by the dose delivery mechanism of the inhalation device 100. When incorporated using the example dose delivery mechanism described herein, the switch 130 may be actuated by a slider 140 as the mouthpiece cover 108 is moved from a closed position to an open position. Although it should be appreciated that if the inhalation device 100 includes a different dose delivery mechanism, then the switch 130 may be actuated by a different component of the dose deliver mechanism. When the switch 130 is actuated, the electronics module 120 may generate a signal causing the electronics module 120 to change states, such as from an off or sleep state to an active state. When in the active state, the controller of the electronics module 120 may wake and provide power to the sensor system 128 to enable the sensor system 128 to take measurement readings. Further, the electronics module 120 may store a dosing event (e.g., which may be referred to as a dose delivery event or an actuation event) each time the switch 130 is actuated. As described in more detail below, the electronics module 120 may have a plurality of power states, each with respective power consumption levels. For example, the electronics module 120 may be configured to operate in a system off state, a sleep state, and/or an active state, where the electronics module 120 consumes the least amount of power while in the off state (e.g., no power or just enough to run a clock), the sleep state uses more power than the off state (e.g., to drive the memory, the communication circuit, and/or a timer or clock), and the active state uses the most amount of power (e.g., to drive the controller, one or more sensors, the communication circuit, potentially in a faster advertising mode than the sleep state, and/or a timer or clock).

The sensor system 128 may include one or more sensors, such as one or more pressure sensors, temperature sensors, humidity sensors, orientation sensors, acoustic sensors, and/or optical sensors. The pressure sensor(s) may include a barometric pressure sensor (e.g., an atmospheric pressure sensor), a differential pressure sensor, an absolute pressure sensor, and/or the like. The sensors may employ microelectromechanical systems (MEMS) and/or nanoelectromechanical systems (NEMS) technology. The pressure sensor(s) may be configured to detect and/or measure pressure changes within the inhalation device 100 caused by an inhalation from (or an exhalation into) the mouthpiece 106. One or more of the measured pressure changes may be used to determine the amount of airflow (e.g., an airflow rate) through the flow pathway 119 of the mouthpiece 106. The magnitude of the airflow may indicate whether a user is properly using the device 100. For example, if the deagglomerator 121 is configured to aerosolize a dose of medication when the airflow rate exceeds a threshold, a use may be deemed compliant if the determined airflow rate is above the threshold. Conversely, the use may be deemed non-compliant if the determined airflow is below the threshold. Examples of the sensors are described in reference to US 2020/0360630 A1, the entire disclosure of which are incorporated herein by reference. Further, it should be appreciated that the controller of the electronics module 120 may be configured to convert pressure measurements received from the pressure sensor to a flow rate based on Bernoulli's principle (e.g., the Bernoulli/Venturi effect).

The controller of the electronics module 120 may receive signals corresponding to measurements from the sensor system 128. The electronics module 120 (e.g., and/or a mobile application residing on an external device) may use measurements from the sensor system 128 to determine one or more dosing events. For example, the electronics module 120 may be configured to compare one or more measurements from the sensor system 128 to one or more threshold values to categorize an inhalation event as a no/low inhalation event, a fair inhalation event, a good inhalation event, an excessive inhalation event, and/or an exhalation event. For example, the electronics module may generate a good inhalation event when the measurements from the sensor system 128 indicate a flow rate in a particular range (e.g., between 200 liters per min (L/min) and 45 L/min), generate a fair inhalation event when the measurements from the sensor system 128 indicate a flow rate in another range (e.g., 30 L/min and 45 L/min), generate a no inhalation event when the measurements from the sensor system 128 indicate a flow rate that is less than a threshold value (e.g., 30 L/min), and an excessive inhalation event when the measurements from the sensor system 128 indicate a flow rate that is greater than an upper threshold (e.g., greater than 200 L/min).

The temperature sensor(s) may include a thermistor, a thermocouple, a resistance temperature detector, a temperature sensor chip and the like. The temperature sensor(s) may be configured to provide a temperature reading to the controller of the electronics module 120 and/or aggregated temperature readings over time. The temperature sensor(s) may be configured to measure the external temperature in the space proximate to the inhalation device 100. Accordingly, main housing 104 and/or the top cap 102 may include an opening (e.g., a vent) to allow for the temperature sensor(s) to measure the ambient temperature external to the housing.

Alternatively or additionally, the temperature sensor(s) may be configured to measure temperature within the inhalation device 100, such as within one or more of the top cap 102, the main housing 104, and/or the mouthpiece 106 of the inhalation device 100. The ability to measure both internal and external temperature may allow the electronics module 120 to determine the operating temperature of the components of the electronics module 120, the temperature of the air flowing through the inhalation device 100 when a user inhales through the inhalation device 100, etc. Accordingly, the electronics module 120 may be configured to detect an over or under temperature condition, such as an over temperature condition of one or more of the components of the electronic module 120 (e.g., such as another sensor, like a pressure sensor), an over temperature condition of the inhalation device 100, an ambient temperature that exceeds a threshold, etc. The electronics module 120 may be configured to cause the communication circuit 129 to transmit a temperature message to an external device (e.g., a mobile device) that indicates an over temperature condition, an ambient temperature reading, and/or a temperature reading of internal to the inhalation device 100 (e.g., such as a temperature change detected through the flow channel of the inhalation device 100).

Further, in some examples, the pressure sensor may include a temperature sensor. The humidity sensor(s) may include a capacitive sensor, a resistive sensor, a thermal conductivity sensor and the like. The humidity sensor(s) may be configured to provide a humidity reading to the controller of the electronics module 120 and/or aggregated humidity readings over time. The temperature and/or humidity measurements may be used to identify and track the environmental conditions in which the inhalation device 100 is used or stored. The temperature and/or humidity measurements may be used to determine whether the device 100 is being operated or stored in an environment that could compromise the proper operation of the device 100 and/or the efficacy of the medication in the medication reservoir 110. For example, extreme hot or cold temperatures, or excessive humidity levels, may contribute to device failures and/or alter the properties of the medication in the reservoir 110.

The orientation sensor(s) may include an accelerometer, a gravity (G) sensor, a gyroscope, a magnetometer and the like. The orientation sensor(s) may be configured to provide an orientation reading (e.g., acceleration, rotation, direction, etc.) to the controller of the electronics module 120 and/or aggregated orientation readings over time. The data from the orientation sensor(s) may be used to identify and track how a user is handling or interacting with the device 100 when attempting to receive a dose of medication. The data may be used to determine whether the device 100 is being operated in a compliant manner, such as during dose delivery. For example, the orientation sensor(s) may indicate whether a user is holding the device 100 upside down during inhalation, which may prevent or impede the delivery of a full dose of medication from the dosing cup 116 and/or the dosing chamber 117.

As noted above, the electronics module 120 may include one or more indicators, such as an LED, which may be housed or located on the device 100 such that any provided feedback may be observed by a user. The controller in the electronics module 120 may operate the indicators to provide feedback to users regarding their use of the inhalation device 100 and/or the conditions under which the inhalation device 100 is being used or stored. The controller may cause the status of the indicators to change (e.g., the LED may turn on, flash, change color, etc.) if one or more measurements from the sensor system 128 are above or below a predetermined threshold. For example, the controller may cause the LED may illuminate if the measured change in pressure, the determined airflow rate, the measured temperature and/or the measured humidity level exceeds the threshold. The controller may cause the LED may illuminate if the data from the orientation sensor(s) indicates that the device 100 is not being held properly.

The data from the sensor system 128 (e.g., pressure change, temperature, humidity level, etc.) and/or parameters derived therefrom (e.g., airflow rate) may be communicated to an external device, such as a smartphone, tablet or computer. More specifically, the wireless communication circuit 129 in the electronics module 120 may include a transmitter and/or receiver (e.g., a transceiver), as well as additional circuitry. For example, the wireless communication circuit 129 may include a Bluetooth chip set (e.g., a Bluetooth Low Energy chip set), a ZigBee chipset, a Thread chipset, etc. As such, the electronics module 120 may wirelessly provide data to the external device for review and/or additional processing. The external device may include software for processing the received information and for providing compliance and adherence feedback to users of the inhalation device 100 via a graphical user interface (GUI).

The power supply 126 may provide power to the electrical components of the PCB 122. The power supply 126 may be any suitable source for powering the electronics module 120, such as a coin cell battery, for example. The power supply 126 may be rechargeable or non-rechargeable. The power supply 126 may be housed by the power supply holder 124. The power supply holder 124 may be secured to the PCB 122 such that the power supply 126 maintains continuous contact with the PCB 122 and/or is in electrical connection with the components of the PCB 122.

The selection of the power supply 126 may be based various factors, such as its size, weight, cost and/or power capacity. Basing the selection of the power supply 126 on one attribute may negatively affect the operation or design of the inhalation device 100 with regard to other attributes of the power supply 126. For example, a supply with the smallest physical dimensions, lowest weight, and/or lowest cost may have insufficient capacity to power the electronics module 120 for a desired period (e.g., the normal operating life of the device 100). Conversely, a supply with sufficient capacity to power the electronics module 120 for the desired period may not fit within the space available in the top cap 102 and/or may be more expensive. Accordingly, the selection of the power supply 126 may include balancing one or more of its technical and/or commercial attributes. In addition, the operation of the electronics module 120 may be configured to limit or manage the power consumption from the power supply 126, which may enable the selection of a smaller, less expensive supply that can reliably power the electronics module 120 for the desired period and under the desired operating conditions.

The electronics module 120 may have a plurality of power states, each with respective power consumption levels. For example, the electronics module 120 may be configured to operate in a system off state, a sleep state, and/or an active state. While the electronics module 120 is in the active state, the electronics module 120 may operate in one or more modes, such as a measurement mode, a data storage/data processing mode, an advertising mode, and/or a connected mode. It will be appreciated that the electronics module 120 may operate in multiple modes at one time (e.g., the modes may overlap).

In the measurement mode, the controller of the electronics module 120 may power on the sensor system 128. The controller may cause the sensor system 128 to take pressure measurement readings, temperature readings, humidity readings, orientation readings, etc. for a predetermined period (e.g., up to 60 seconds) and/or until the mouthpiece cover 108 is closed or no changes in measurements are detected. The controller may turn off one or more components of the electronics module 120 while the sensor system 128 is capturing readings to further conserve power. The sensor system 128 may sample the readings at any suitable rate. For example, the sensor system 128 may have a sample rate of 100 Hz and thus a cycle time of 10 milliseconds. The sensor system 128 may generate a measurement complete interrupt after the measurement cycle is complete. The interrupt may wake the controller or cause it to turn on one or more components of the electronics module 120. For example, after or while the sensor system 128 is sampling one or more pressure measurements, temperature readings, humidity readings, orientation readings, etc., the controller may process and/or store the data and, if measurements are complete, power off the sensor system 128.

In the data storage/data processing mode, the controller may power on at least a portion of the memory within the electronics module 120. The controller may process the readings from the sensor system 128 to compute, estimate, or otherwise parameters (e.g., usage and/or storage conditions) and store the parameters in memory. The controller may also compare the readings and/or parameters to one or more thresholds or ranges to assess how the inhalation device 100 is being used and/or the conditions under which the device 100 is being used. Depending on the results of the comparison, the controller may drive the indicators (e.g., an LED) to provide feedback to the user of the inhalation device 100. As noted above, the electronics module 120 may operate in the measurement mode and the data storage/data processing mode simultaneously.

In the connected mode, the communication circuit and memory may be powered on and the electronics module 120 may be connected to or “paired” with an external device, such as a smartphone. The controller may retrieve data from the memory (e.g., sensor data and/or parameters derived from the sensor data) and wirelessly transmit the data to the external device. The controller may retrieve and transmit all of the data currently stored in the memory. The controller may also retrieve and transmit a portion of the data currently stored in the memory. For example, the controller may be able to determine which portions have already been transmitted to the external device and then transmit the portion(s) that have not been previously transmitted. Additionally or alternatively, the external device may request specific data from the controller, such as any data that has been collected by the electronics module 120 after a particular time or after the last transmission to the external device. The controller may retrieve the specific data, if any, from the memory and transmit the specific data to the external device.

Further, when connected with the external device, the electronics module 120 may be configured to transmit Bluetooth special interest group (SIG) characteristics for managing access to data stored in the module 120. The Bluetooth SIG characteristics may include one or more of a manufacturer name of the inhalation device 100, a serial number of the inhalation device 100, a hardware revision number of the inhalation device 100, and/or a software revision number of the inhalation device 100. When connected with the external device, the electronics module 120 may retrieve data from memory and transmit the data to the external device.

The data stored in the memory of the electronics module 120 (e.g., the signals generated by the switch 130, the measurement readings taken by the sensor system 128 and/or the parameters computed by the controller of the electronics module 120) may be transmitted to an external device, which may process and/or analyze the data to determine the usage parameters associated with the inhalation device 100. The data may include any a usage parameter (e.g., usage event), which for example, may include or indicate a use of the respective inhaler. In a relatively simple implementation, the at least one value may comprise “TRUE” when use of, for example an inhalation using, the respective inhaler has been determined, or “FALSE” when no such use of the respective inhaler is determined. However, the usage parameters may include any combination of the events described herein, such as, no inhalation events, low inhalations events, good inhalation events, excessive inhalation events, exhalation events, actuation events, error events, underuse events, overuse events, etc. Further, the usage parameters may include a count of the number of uses of the inhalation device 100, a measure of airflow of inhalation device 100, other measurements indicating the usage of the medicament of inhalation device 100, such as the actuation of a switch configured to detect usage of inhalation device 100 (e.g., when the mouthpiece cover is moved from a closed position to an open position and/or a switch that is actuated upon the priming of the inhaler, such as to move and/or open a blister of medicament comprised within the inhaler), feedback from one or more sensors configured to detect use of inhalation device 100, and/or the actuation of one or more buttons configured to be depressed upon use of inhalation device 100.

Further, it should be appreciated that the electronics module (e.g., the processor) 120 is configured to timestamp the data (e.g., associate a timestamp with the data). For example, the electronics module 120 may include a local mean time clock, and may associate a timestamp that indicates the local mean time of the inhalation device 100 with the data determined by the inhalation device 100. In other examples, the electronics module 120 may operate as an internal counter. When operating as an internal counter, the electronics module 120 determines a relative count (e.g., as opposed to providing a mean solar time, such as a local mean time), and associates the relative count with the determined data. For instance, the electronics module 120 may start an internal counter (e.g., which counts up from 0 indefinitely) when, for example, the electronics module 120 is woken out of an energy-saving sleep mode for the first time (e.g., after the mouthpiece cover is opened for the first time). Thereafter, any timestamp generated by the electronics module 120 may be a relative time (or count) based on the internal counter. The electronics module 120 may periodically update the system clock every 250 microseconds (μs).

Further, a software application residing on the external device may generate feedback for the user based on data received from the electronics module 120. For example, the software application may generate daily, weekly, or monthly report, provide confirmation of error events or notifications, provide instructive feedback to the user, and/or the like.

FIG. 5 is a diagram of an example system 500 including the inhalation devices 501a, 501b, mobile devices 502a, 502b, 502c, a public and/or private network 508 (e.g., any combination of the Internet, a cloud network, and/or the like), and a computer (e.g., a server) 512 associated with a health care provider, such as a hospital or hospital system, a health system, a medical group, a physician, a clinic, and/or a pharmaceutical company.

The system 500 also includes a digital health platform (DHP) 510 that resides on one or more servers, and may include computer software configured to perform the functions described in relation to the DHP 510. The DHP 510 may comprise memory. The memory of the DHP 510 may comprise a computer-readable storage media or machine-readable storage media that maintains computer-executable instructions for performing one or more as described herein. For example, the memory may comprise computer-executable instructions or machine-readable instructions that include one or more portions of the procedures described herein. The DHP 510 may access the instructions from their respective memory for being executed to cause the processor(s) of the DHP 510 to operate as described herein. The memory may comprise computer-executable instructions for executing configuration software. For example, the computer-executable instructions may be executed to perform, in part and/or in their entirety, one or more procedures, such as the procedures 600, 700, 800, and/or 900 as described herein. Further, the memory may have stored thereon one or more settings and/or control parameters associated with the DHP 510.

The mobile devices 502a, 502b, 502c may include a smart phone (e.g., an iPhone® smart phone, an Android® smart phone, or a Blackberry® smart phone), a personal computer, a laptop, a wireless-capable media device (e.g., MP3 player, gaming device, television, a media streaming devices (e.g., the Amazon Fire TV, Nexus Player, etc.), etc.), a tablet device (e.g., an iPad® hand-held computing device), a Wi-Fi or wireless-communication-capable television, a wearable device (e.g., the Apple Watch®), or any other suitable Internet-Protocol-enabled device. The mobile devices 502a, 502b, 502c may include a processor, memory, a communication circuit (e.g., a transceiver), speakers, microphone, and/or a display screen. The mobile devices 502a, 502b, 502c may have stored thereon a mobile application that is configured to cause the mobile device to perform the functions described herein, such as communicate with one or more inhalation devices 501a, 501b and/or the DHP 510, receive, process, and/or aggregate the data received from the inhalers, generate new data and/or alerts based on the data received from the inhalers, and/or generate feedback (e.g., alerts), such as notifications, GUIs, or audio feedback, based on the inhaler data.

The mobile devices 502a, 502b, 502c may comprise memory. The memory of each mobile device 502a, 502b, 502c may comprise a computer-readable storage media or machine-readable storage media that maintains computer-executable instructions for performing one or more as described herein. For example, the memory may comprise computer-executable instructions or machine-readable instructions that include one or more portions of the procedures described herein. The mobile devices 502a, 502b, 502c may access the instructions from their respective memory for being executed to cause the controller of the mobile devices 502a, 502b, 502c to operate as described herein. The memory may comprise computer-executable instructions for executing configuration software. For example, the computer-executable instructions may be executed to perform, in part and/or in their entirety, one or more procedures, such as the procedures 600, 700, 800, and/or 900 as described herein. Further, the memory may have stored thereon one or more settings and/or control parameters associated with the mobile devices 502a, 502b, 502c.

Also provided is a computer program comprising computer program code stored on the memory of the mobile devices 502a, 502b, 502c which is adapted, when the computer program is run on the mobile devices 502a, 502b, 502c, to implement any of the methods and technique described herein. In a preferred embodiment, the computer program takes the form of the mobile application, at least partially, for example the mobile application residing on one or more of the mobile devices 502a, 502b, 502c. And in some examples, the computer program is provided partially on memory of the mobile application and partially on memory of the DHP 510. The embodiments described herein for the system 500 are applicable to the method and the computer program. Moreover, the embodiments described for the method and computer program are applicable to the system 500.

Although described as mobile devices, the system 500 may, in some examples, include stationary devices or a combination of mobile devices and stationary devices. The stationary devices include smart home interface devices, such as smart speakers, smart displays, smart home automation devices, and/or the like. The stationary devices include similar hardware and/or software as the mobile devices described herein (e.g., a processor, memory, a communication circuit (e.g., a transceiver), speakers, microphone, and/or a display screen), and therefore, are configured to perform the functions described herein with respect to the mobile devices.

The mobile devices 502a, 502b, 502c may be configured to communicate with the inhalation devices 501a, 501b. The mobile devices 502a, 502b, 502c may also be configured to communicate with the public and/or private network 508, which may be in communication with the DHP 510 and/or a computer 512 associated with a health care provider. For example, the mobile devices 502a, 502b, 502c may include communication circuit (e.g., a transceiver), and as such may be configured to transmit and/or receive RF signals via a Wi-Fi communication link, a Wi-MAX communications link, a Bluetooth® or Bluetooth Smart communications link, a near field communication (NFC) link, a cellular communications link, a television white space (TVWS) communication link, or any combination thereof. The mobile devices 502a, 502b, 502c may transfer data through the public and/or private network 508 to the DHP 510 using, for example, a dedicated API. For example, the mobile devices 502a, 502b, 502c may send inhaler data associated with one or more of the inhalation devices 501a, 501b to the DHP 510.

The inhalation device 501a may comprise an inhaler with medicament. The inhalation device 501b may comprise a demonstrator inhaler. The inhalation device 501b may be an inhaler with no medicament. For example, the inhalation device 501b may be an exact replica of the inhalation device 501a without medicament. A user may use a demonstrator inhaler to practice inhalation. It should be appreciated that although the system 500 is shown with each of the mobile devices 502a, 502b, 502c paired with two inhalation devices 501a, 501b, the system 500 is not limited to this configuration. Instead, each of the mobile devices 502a, 502b, 502c may be paired with one or more demonstrator inhalers (e.g., such as inhalation device 501b) and/or one or more inhalers with medicament (e.g., such as inhalation device 501a).

The inhalation devices 501a, 501b may include a communication circuit (e.g., such as the communication circuit 129), such as a Bluetooth radio, for transferring data to an external device (e.g., one or more of the mobile devices 502a, 502b, 502c). The data may be referred to as inhaler data, usage data, usage parameters, event records, and/or usage events. The data may include any of the data described herein, such as the signals generated by a switch (e.g., the switch 130), the measurement readings taken by a sensor (e.g., the sensor system 128), and/or parameters computed by the controller of an electronics module (e.g., the electronics module 120). The data may include any combination of inhaler events, no inhalation events, low inhalations events, good inhalation events, excessive inhalation events, exhalation events, actuation events, error events, underuse events, overuse events, etc. The data may include a count of the number of uses of the respective inhalation device 501a, 501b, a measure of airflow of the respective inhalation device 501a, 501b, other measurements indicating the usage of the medicament of inhalation device 501a, such as the actuation of a switch configured to detect usage of the respective inhalation device 501a, 501b (e.g., when the mouthpiece cover is moved from a closed position to an open position and/or a switch that is actuated upon the priming of the inhaler, such as to move and/or open a blister of medicament comprised within the inhaler), feedback from one or more sensors configured to detect use of the respective inhalation device 501a, 501b, and/or the actuation of one or more buttons configured to be depressed upon use of the respective inhalation device 501a, 501b. The data may be associated with a timestamp, for example, as described herein.

The inhalation devices 501a, 501b may receive data from the mobile devices 502a, 502b, 502c, such as, for example, program instructions, operating system changes, dosage information, alerts or notifications, acknowledgments, etc. Further, although illustrated as a two inhalation devices 501a, 501b per mobile device 502a, 502b, 502c, the system 500 may include any number of inhalation devices 501a, 501b that are associated with a user. Additionally or alternatively, although illustrated as three mobile devices 502a, 502b, 502c each associated with a respective user, the system 500 may include any number of mobile devices 502a, 502b, 502c associated with a plurality of different users. It should be noted that some users will have multiple inhalation devices 501a that include the same medicament. For example, a user may have multiple inhalation devices 501a that include a rescue medicament (e.g., and keep them at different locations). Further, a user may have multiple inhalation devices 501a that include a particular maintenance medicament, such as when they are transitioning between refills. Further the system 500 is configurable with the inhalation devices 501a, 501b of a plurality of different users. As such, the system 500 is configured to communication, via respective mobile devices, with a plurality of different inhalers that are associated with a plurality of different users.

The mobile devices 502a, 502b, 502c may process and analyze the data to determine the usage parameters associated with the respective inhalation device 501a, 501b. For example, the mobile devices 502a, 502b, 502c may process the data to identify inhaler events, usage events, such as no inhalation events, low inhalations events, good inhalation events, excessive inhalation events, exhalation events, actuation events, error events, underuse events, overuse events, etc. The mobile devices 502a, 502b, 502c may include a display device and software for visually presenting the usage parameters and/or data related to usage events through a GUI on the display device.

The mobile devices 502a, 502b, 502c may be configured to receive data (e.g., usage events) and associated timestamps (e.g., a relative count from an internal counter of the electronics module 120) from the inhalation devices 501a, 501b. The mobile devices 502a, 502b, 502c may determine a local mean time and a time zone for a timestamp, and associate the local mean time and time zone with the data (e.g., usage event). The mobile devices 502a, 502b, 502c may then send the data and the associated local mean time and time zone to the DHP 510. The DHP 510 may associate the data, local mean time, and time zone with a user. Alternatively or additionally, the mobile devices 502a, 502b, 502c may associate the data, local mean time, and time zone with a user, and/or the DHP 510 may determine the local mean time based on the timestamp received from the inhalation device 501a, 501b.

The inhalation devices 501a, 501b may include a barcode, such as a Quick Response (QR) code (e.g., such as the QR code 160 shown in FIG. 1) that is used to facilitate the pairing process between the inhalation devices 501a, 501b and a mobile device (e.g., any of the mobile devices 502a, 502b, 502c). For instance, in some examples, the inhalation devices 501a, 501b do not include an actuator, button, or switch to initiate a pairing process with a mobile device, and as such, the QR code may be used. Although described as a QR code, other types of barcodes may be used. The use of the QR code to initiate the pairing process may further reduce the required battery/power consumption of the electronics module of the inhalation devices 501a, 501b. Further, although the QR code is illustrated as being located on the top of a top cap (e.g., such as the top cap 102 shown in FIGS. 1-4) of the inhalation devices 501a, 501b, in other examples, the inhalation devices 501a, 501b may include a QR code that is located elsewhere on the inhalation devices 501a, 501b, such as on a main housing (e.g., such as the main housing 104 shown in FIGS. 1-3) or on a mouthpiece cover (e.g., such as the mouthpiece cover 108 shown in FIGS. 1-3). The mobile devices 502a, 502b, 502c may include a camera, and the mobile devices 502a, 502b, 502c may be configured to access the camera and read the QR code.

The QR code may include (e.g., be coded to indicate) various types of information associated with the inhalation devices 501a, 501b. The QR code may include a BLE passkey that is unique to the inhalation devices 501a, 501b. Upon reading or scanning the QR code using the camera, the mobile device may determine the BLE passkey associated with the respective inhalation device 501a, 501b and complete an authentication process, thereby enabling it to communicate with the electronics module using the BLE passkey. If the communications session is subsequently lost because, for example, the inhalation device 501a, 501b moves out of range, the mobile device 502a, 502b, 502c may be configured to use the BLE passkey to automatically pair with the electronics module without using the QR code when the inhalation device 501a, 501b is back within range.

Further, in some examples, the QR code may include an indication of the type of the inhalation device 501a, 501b (e.g., the medication type, the number of doses, the strength, the dosing schedule, etc.). Table 1 provides a non-limiting example of the identifiers included in the QR code for various inhalation devices 501a, 501b.

TABLE 1

| | | | | Total dose | |
|---|---|---|---|---|---|
| | | | | count of | |
| Identifier | Brand | | Dose | inhaler | Medicament |
| in QR | of | | strength | prior | identification |
| code | inhaler | Medicament | (mcg) | to use | number |
| AAA200 | ProAir | albuterol | 117 | 200 | AAA200 |
| | Digihaler | | | | |
| AAA030 | ProAir | albuterol | 117 | 30 | AAA030 |
| | Digihaler | | | | |
| FSL060 | AirDuo | fluticasone/ | 55/14 | 60 | FSL060 |
| | Digihaler | salmeterol | | | |
| FSM060 | AirDuo | fluticasone/ | 113/14 | 60 | FSM060 |
| | Digihaler | salmeterol | | | |
| FSH060 | AirDuo | fluticasone/ | 232/14 | 60 | FSH060 |
| | Digihaler | salmeterol | | | |
| FPL060 | ArmonAir | fluticasone | 55 | 60 | FPL060 |
| | Digihaler | | | | |
| FPM060 | ArmonAir | fluticasone | 113 | 60 | FPM060 |
| | Digihaler | | | | |
| FPH060 | ArmonAir | fluticasone | 232 | 60 | FPH060 |
| | Digihaler | | | | |

As shown in Table 1, the identifier further denotes the dose strength and the total dose count of the inhalation device 501a, 501b prior to use. The mobile device 502a, 502b, 502c may use the information identifier by the identifier to, in combination with the usage information, control a user interface of the mobile device 502a, 502b, 502c to issue a notification when the label recommended dosages have been exceeded, as previously described. Alternatively or additionally, the mobile device 502a, 502b, 502c may use the total dose count of the inhalation device 501a, 501b prior to use and the usage information to determine the number of doses remaining in the respective inhalation device 501a, 501b.

The QR code on the inhalation device 501a, 501b may, for instance, further comprise a security key, for example in the form of a series of alphanumerical characters, for preventing unauthorized users from accessing the respective inhalation device 501a, 501b. The mobile device 502a, 502b, 502c may be able to decrypt the respective encrypted data once the mobile device 502a, 502b, 502c has been provided with the security key, but may not be able to decrypt the respective encrypted data before the mobile device 502a, 502b, 502c has been provided with the security key. More generally, the security key may be included in the respective identifier.

In a non-limiting example, the system 500 may be configured to restrict one or more, e.g., each, of the inhalers 501a, 501b associated with a respective one of the mobile devices 502a, 502b, 502c in the system 500 to a single user account. In such an example, a passkey, e.g., provided in the QR code, may allow synchronization between the respective inhalation devices 501a, 501b and mobile applications of the system 500. The passkey and, in turn, the usage parameter data, e.g., inhalation and/or usage data, from the respective inhalation devices 501a, 501b may be public. This public inhalation data may not be associated with the particular subject until synchronization with the single user account. Since, in some examples, the system 500 may be configured to restrict the respective inhalation device 501a, 501b to being associated with the single user account, the respective inhalation device 501a, 501b may be prevented from being synchronized with another user account, for example in situations where the inhalation device 501a, 501b is lost or stolen. In this way, third parties may be prevented from acquiring usage parameter data which is not theirs.

The mobile application (e.g., memory that stores executable instructions, for example, that may be comprised within a mobile device 502a, 502b, 502c and/or the DHP 510) can determine the type of inhalation device 501a, 501b when receiving the QR code (e.g., the medicament type, the dosage strength, and the number of doses), for example, prior to the first use of the inhalation device 501a, 501b by a user. For example, the mobile device 502a, 502b, 502c may receive (e.g., capture) an image of the QR code using the camera of the mobile device 502a, 502b, 502c. The mobile device 502a, 502b, 502c may then decode the image of the QR code to acquire the data stored within the QR code. In some examples, the QR code may comprise a multi-digit alphanumeric code, such as a six-digit code (e.g., ssm060, aaa200, etc.) that indicates the type of the inhalation device 501a, 501b. For example, the multi-digit alphanumeric code may be a unique drug product identifier (e.g., product ID) of the inhalation device 501a, 501b. Accordingly, the QR code may directly communicate the type of the inhalation device 501a, 501b (e.g., the medication type, the number of doses, the strength, the dosing schedule, etc.) via the multi-digit alphanumeric code provided via the QR code (e.g., and as such, the mobile application does not have to access a website using the QR code to acquire the type of the inhalation device 501a, 501b). A multi-digit alphanumeric code of “AAA030” may, for example, indicate that the medication type is albuterol, the strength is 117 mcg, and the number of doses is 30.

In some examples, the BLE passkey provided via the QR code may comprise the multi-digit alphanumeric code, for example. Further, in some embodiments, the QR code may not directly indicate, to the mobile device 502a, 502b, 502c, the information relating to the medication type, the number of doses, the strength, the dosing schedule, etc. of the inhalation device 501a, 501b. Rather, the QR code may comprise information that can be used by the mobile device 502a, 502b, 502c to acquire such information relating to the inhalation device 501a, 501b from a remote device (e.g., a cloud-based system, such as a remote server).

Moreover, in some examples, the QR code may include any combination of a serial number of the inhalation device 501a, 501b, a hardware revision number of the inhalation device 501a, 501b, and/or a software revision number of the inhalation device 501a, 501b. Further, although described with reference to a QR code, the inhalation device 501a, 501b may include any code (e.g., barcode) that indicates the type of the inhalation device 501a, 501b, a communication passkey (e.g., BLE passkey), a manufacturer name of the inhalation device 501a, 501b, a serial number of the inhalation device 501a, 501b, a hardware revision number of the inhalation device 501a, 501b, a software revision number of the inhalation device 501a, 501b, and/or the like.

Upon receiving the QR code, the mobile device 502a, 502b, 502c may determine the details of the inhalation device 501a, 501b (e.g., the medication type, the number of doses, the strength, the dosing schedule, etc.), such as directly through a multi-digit alphanumeric provided via the QR code. Alternatively or additionally, in some example, the mobile device 502a, 502b, 502c may be configured to send a request (e.g., that includes the multi-digit alphanumeric code) to a DHP 510 for the details relating to the inhalation device 501a, 501b (e.g., the medication type, the number of doses, the strength, the dosing schedule, etc.). This can be particular important, for example, in instances where mislabeling issues can arise, and the inhalation device 501a, 501b is mislabeled with the incorrect type of medication. The use of the QR code allows for accurate details relating to the inhalation device 501a, 501b to be acquired by the mobile device 502a, 502b, 502c prior to the user using the inhalation device 501a, 501b, for example. This may, for example, help prevent issues relating to incorrect dosage reminders based on incorrect dosing schedules, incorrect refill warning, etc. that could otherwise have been determined based on the incorrect medication labeling on the inhalation device 501a, 501b.

Further, in some examples, the mobile device 502a, 502b, 502c may determine that the user associated with the mobile device 502a, 502b, 502c is not prescribed the medication and/or dose strength indicated by the QR code. For instance, the mobile device 502a, 502b, 502c may send the medication type or the strength of the inhaler indicated by the QR code to the DHP 510, and in response, may receive an indication of the user's compatibility with the medication type or the strength of the inhalation device 501a, 501b from the cloud-based server (e.g., such as a direct compatible or not compatible message, or an indication of the medication types and strengths associated with the user). For example, the mobile device 502a, 502b, 502c may request and receive the user's prescription information from the DHP 510. And the mobile device 502a, 502b, 502c may generate an alert (e.g., a GUI displayed on the mobile device 502a, 502b, 502c and/or at the computer 512 associated with the HCP) that indicates that the user has the incorrect inhalation device 501a, 501b, either based on the medicament type or strength, as indicated by the QR code. Further, in such instances, the mobile device 502a, 502b, 502c may reject the pairing process with the inhalation device 501a, 501b.

Further, in some examples, the mobile application may be used with specific types of inhalation devices, but not all. Accordingly, the mobile device 502a, 502b, 502c may display an error message if the information provided by the QR code indicates that that the inhalation device 501a, 501b is not compatible with the mobile application. If, for example, the mobile device 502a, 502b, 502c determines that the inhalation device is not compatible with the mobile application (e.g., based on the QR code or multi-digit alphanumeric code), the mobile device 502a, 502b, 502c may reject the pairing process of the inhalation device 501a, 501b with the mobile device 502a, 502b, 502c (e.g., before data transfer or a first use of the inhalation device 501a, 501b). If the mobile device 502a, 502b, 502c determines that the inhalation device 501a, 501b is compatible with the mobile application (e.g., based on the QR code or multi-digit alphanumeric code), the mobile application may accept or allow the pairing process of the inhalation device 501a, 501b with the mobile device 502a, 502b, 502c.

In some examples, the QR code may provide a link to a mobile application store to download the correct mobile application for the type of inhalation device 501a, 501b. This can help to reduce the burden on the user to manually find, select, download, and install the correct mobile application. In the process, it can help to avoid installation of a mobile application that is not the correct application (or not the correct version of the application) for the type of inhalation device in question.

The DHP 510 may be configured to receive and aggregate inhaler data (e.g., usage events) from inhalation devices 501a, 501b that are associated with a plurality of different users. In some examples, the DHP 510 may reside on or across one or more servers, and may include computer software configured to perform the functions described in relation to the DHP 510. For example, the DHP 510 may include a dashboard application that may be accessible by the computer 512 associated with a health care provider, such as a hospital or hospital system, a health system, a medical group, a physician, a clinic, and/or a pharmaceutical company. In some examples, the dashboard application is a web application (e.g., a web portal). For example, the DHP 510 may also be configured to provide data, such as inhaler data, to clinicians and physicians through the use of the dashboard application (e.g., via a REST API).

The DHP 510 is configured to receive and aggregate data from inhalation devices 501a, 501b, where the inhalation devices 501a, 501b may be associated with a plurality of different users. For example, the DHP 510 is configured to receive and store inhaler data from the mobile devices 502a, 502b, 502c (e.g., the patient-facing mobile applications). The inhaler data may include any of the data described with reference to the inhalers described herein, such as inhaler events, usage events, error events, inhalation profiles, associated timestamps, medicament types, etc. The DHP 510 is configured to analyze and manipulate the data. For example, the DHP 510 may aggregate the data across a plurality of the inhalation devices 501a, 501b that it receives data from, and then the DHP 510 may analyze the aggregated data, for example, to determine one or more metrics, provide feedback, etc. Further, the DHP 510 is also configured to provide data (e.g., or analytical information based on the data) to the user (e.g., via mobile devices 502a, 502b, 502c) or to the computer 512 associated with a health care provider (e.g., via the dashboard application). The inhaler data may include any of the data described with reference to the inhalation devices described herein.

The inhaler data may be associated with an inhalation device 100 and/or a user profile, for example, at the mobile devices 502, 504, 506 and/or at the DHP 510. One user profile may be associated with multiple inhalation devices 100 of the same and/or different medicament types. The DHP 510 may also de-identify (e.g., disassociate) the inhaler data with a particular user profile, and the DHP 510 may perform analytics on de-identified data relating to the inhalation devices 100. Although described as receiving the inhaler data from the mobile devices 502, 504, 506, in some examples, the DHP 510 may receive the data directly from the inhalation devices 501a, 501b themselves, such as in instances where the communication circuits of the inhalation devices 501a, 501b include cellular chipsets that are capable of communicating directly with the DHP 510.

The DHP 510 may cause the computer 512 associated with the health care provider to provide inhaler data to practitioners and health care professionals to allow them to view inhaler data specific to a program to which a patient has consented. In one example, the DHP 510 causes the computer 512 associated with the health care provider to provide the inhaler data via a graphical user interface (GUI) that is presented on a display device associated with the health care provider's computer.

The DHP 510 may define any number of programs, which in some instances may be configured and altered by a health care provider. When providing inhaler data to a health care provider, the DHP 510 may generate an alert (e.g., generate and provide a GUI) that is specific to a particular program associated with that health care provider. A program defines a set of criteria, such as types of medications (e.g., any combination of rescue and/or maintenance medications), specific patients and in turn their applicable inhalers, other users of the programs such as particular physicians, practice groups, and/or administrators, the types of data presented to the health care provider such as charts, event tables, usage summaries, etc. The health care provider may configure and establish any number of programs using the DHP 510. Further, a particular patient and their inhalers may be associated with any number of unique programs. In some examples, the programs are stored and maintained by the DHP 510, and the computer 512 associated with the health care provider is configured to access the data relevant to each program from the DHP 510 using, for example, an application, such as a dashboard or web application. In such examples, and once a program is established, the DHP 510 is configured to receive inhaler data associated with the program, analyze and manipulate the inhaler data to the extent necessary, and provide program data (e.g., via the dashboard) to the health care provider. The program data may include inhaler data that is specific to the configuration of a particular program, and for example, additional data that is derived from the inhaler data, as is described in more detail below. For example, the DHP 510 may enable a GUI, such as those described herein, on the computer 512 associated with the health care provider that presents the program data to the health care provider.

For example, the DHP 510 may include a dashboard application that may be accessible by the computer 512 associated with a health care provider. In some examples, the dashboard application is a web application (e.g., a web portal). For example, the DHP 510 is configured to provide data, such as inhaler data, to clinicians and physicians through the use of the dashboard application (e.g., via a REST API). The DHP 510 may cause the computer 512 associated with the health care provider to provide inhaler data to practitioners and health care professionals to allow them to view inhaler data specific to a program to which a patient has consented. In one example, the DHP 510 causes the computer 512 associated with the health care provider to provide the inhalation data via a graphical user interface (GUI) that is presented on the health care provider's computer.

The DHP 510 may also employ machine learning and/or predictive modeling techniques. The DHP 510 may comprise one or more machine learning algorithms, such as but not limited to, an instance-based algorithm (e.g., k-Nearest Neighbor (kNN), Learning Vector Quantization (LVQ), Self-Organizing Map (SOM), Locally Weighted Learning (LWL), Support Vector Machines (SVM), etc.), a regression algorithm (e.g., a linear regression algorithm, a logistic regression algorithm, etc.), a decision tree algorithm, a Bayesian algorithm (e.g., a Naive Bayes classifier), an ensemble algorithm (e.g., a weighted average algorithm), etc.

The DHP 510 may train the algorithm using the historical anonymized data received from the inhalers. The DHP 510 may train the algorithm using training data by way of an unsupervised learning method or a supervised learning method, such as, but not limited to a gradient descent or a stochastic gradient descent learning method. An unsupervised learning method may be a type of machine learning that does not use labels for training data. The unsupervised learning method may be a learning method for learning neural networks to identify and classify patterns in the training data itself, rather than correlations between training data and labels corresponding to the training data. Examples of the unsupervised learning method may include clustering and independent component analysis. For example, the unsupervised learning method may include a k-means or a c-means clustering method. A k-means clustering method may group the training data into different clusters, where k defines the number of pre-defined clusters that need to be created. The k-means clustering method may be an iterative algorithm that divides the training data into the k clusters in such a way that each instance of training data belongs to one (e.g., only one) group that has similar properties. A c-means clustering method may group the training data into different clusters, where each instance of training data is assigned a likelihood and/or probability that it belongs to one or more clusters. That is, an instance of training data in a c-means clustering method may belong to a plurality of clusters and is assigned a likelihood for each of the plurality of clusters.

A supervised learning method may use labeled training data to train the machine learning algorithm. As training data is received, the supervised learning method may adjust weights until the machine learning algorithm is appropriately weighted. The supervised learning method may measure the accuracy of the machine learning algorithm using a loss function. The supervised learning method may continue adjusting the weights until the error is reduced below a predetermined threshold. The supervised learning method may comprise gradient boosted decision trees. Gradient boosted decision trees may combine weak learners to minimize the loss function. For example, regression trees may be used to output real values for splits and to be added together. The weak learners may be constrained, for example, to a maximum number of layers, a maximum number of nodes, a maximum number of splits, etc. Trees may be added one at a time to the machine learning algorithm and existing trees may remain unchanged. A gradient descent procedure may be used to minimize loss when adding trees. For example, additional trees may be added to reduce the loss (e.g., follow the gradient). In this case, the additional tree(s) may be parameterized and those parameters may be modified to reduce the loss. The supervised learning method may comprise an XGBoost algorithm. The XGBoost algorithm may comprise an implementation of gradient boosted decision trees that is designed for speed and/or performance. The XGBoost algorithm may automatically handle missing data values, support parallelization of tree construction, and/or continued training.

The training data may include any combination of labeled and unlabeled data. For example, the historical anonymized data received from the inhalers may be an example of labeled data. That is, the data may include any combination of data received from an inhaler or mobile device, or calculated by the DHP 510, such as a day, time, and/or place of an inhalation event, inhalation parameter(s), biometric parameter(s), environmental conditions, daily self-assessment (SA) responses, etc. (e.g., any of the factors described with reference to Table 1). Further, the DHP 510 may identify inhalation parameters, geographic location, and/or environmental conditions of usage events, for example, based on the received data from the inhalers. Further, the DHP 510 may determine that a user experienced an exacerbation event based on manual data entry by the user and/or receipt of data from the health care provider.

The historical anonymized data within the DHP 510 may be input into various machine learning systems and/or predictive models. In response, the machine learning systems and/or predictive models may, based on the inputted anonymized data, be used to assess a user's compliance with a prescribed treatment and/or predict the likelihood of future events, such as the likelihood of a respiratory exacerbations event (e.g., asthma, COPD, or CF exacerbation) or a likelihood of future compliance with the prescribed treatment, as described in more detail herein. For example, the machine learning systems and/or predictive models may be used to detect the attributes, circumstances, and/or conditions (e.g., inhalation parameters, weather conditions, number of recent exacerbations, number or recent rescue and/or maintenance medicament usage events, health related data received from third parties, etc.) that lead to an increased likelihood of exacerbations and/or a lack of compliance for a particular user.

Table 1 is one, non-limiting example, of the particular factors, or attributes, and associated weights that may be applied by the DHP 510 when making a prediction using a predictive model, such as those described herein. As noted herein, the weighting may be a biproduct of the machine learning algorithm, and for example, may indicate the relative significance of the particular factor/attribute when determining the particular score (the user's individualized compliance score, the user's individualized future compliance score, and/or the user's individualized risk score).

TABLE 1
Example Weighting Applied to Factors/Attributes used in a Predictive Model

| Factor/Attribute | | Weighting |
|---|---|---|
| Number of | Normalized* number of rescue inhalations (last 3 days) | 0.104 |
| inhalations | Average number of daily rescue inhalations in the last 5 days | 0.071 |
| | Missed number of maintenance events over the last 3 days | 0.069 |
| | Normalized* number of rescue inhalations today | 0.065 |
| | Normalized* number of inhalation events today | 0.056 |
| | Maximal number of daily rescue inhalations in the last 5 days | 0.051 |
| | Absolute number of rescue inhalations in the last 3 days | 0.045 |
| | Number of rescue inhalations 3 days ago | 0.042 |
| | Number of rescue inhalations 4 days ago | 0.041 |
| | Number of rescue inhalations 2 days ago | 0.026 |
| | Absolute number of inhalation events today | 0.023 |
| | % of change in number of rescue inhalations today, compared to last 3 days | 0.021 |
| | Number of rescue inhalations yesterday | 0.021 |
| | Absolute number of rescue inhalations today | 0.016 |
| | Absolute number of rescue inhalations during night time in the last 3 days | 0.008 |
| | Total weighting: number of inhalations | 0.659 |
| Inhalation | % of change in inhalation peak flow today, compared to last 3 days | 0.082 |
| parameters | % of change in inhalation volume today, compared to last 3 days | 0.051 |
| | Normalized* inhalation peak flow today | 0.046 |
| | Normalized* inhalation volume today | 0.037 |
| | Total weighting: inhalation parameters | 0.216 |
| Biometric | Exacerbations and medical history | 0.012 |
| parameter | Body mass index | 0.009 |
| | Systolic blood pressure | 0.005 |
| | Total weighting: biometric parameter | 0.026 |
| Environmental | Humidity | 0.039 |
| Conditions | Ozone | 0.031 |
| | Particulate Matter | 0.005 |
| | Temperature | 0.014 |
| Daily SA | Self-Assessment Responses | 0.011 |

The electronics module of the inhalation devices 501a, 501b may generate data (e.g., usage events) by comparing signals received from the sensor system and/or the determined airflow metrics to one or more thresholds or ranges, for example, as part of an assessment of how the inhalation devices 501a, 501b are being used and/or whether the use is likely to result in the delivery of a full dose of medication. The electronics module may generate an event record in response to an inhaler event. The generated data for a specific inhaler event may be stored in a respective event record. The inhaler event may comprise actuation of a switch and/or receipt of measurements from a sensor of the sensor system.

For example, where the determined airflow metric corresponds to an inhalation with an airflow rate below a particular threshold, the electronics module may determine that there has been no inhalation or an insufficient inhalation from the mouthpiece of the inhalation device 501a, 501b. If the determined airflow metric corresponds to an inhalation with an airflow rate above a particular threshold, the electronics module may determine that there has been an excessive inhalation from the mouthpiece. If the determined airflow metric corresponds to an inhalation with an airflow rate within a particular range, the electronics module may determine that the inhalation is “good”, or likely to result in a full dose of medication being delivered. The electronics module may associate a timestamp with the data.

The pressure measurement readings and/or the computed airflow metrics may be indicative of the quality or strength of inhalation from the inhalation device 501a, 501b. For example, when compared to a particular threshold or range of values, the readings and/or metrics may be used to categorize the inhalation as a certain type of event, such as a good inhalation event, a low inhalation event, a no inhalation event, or an excessive inhalation event. The categorization of the inhalation may be usage parameters stored as personalized data of the subject. It should be appreciated that inhalation event and inhaler event may be used interchangeably herein.

The no inhalation event may be associated with pressure measurement readings and/or airflow metrics below a particular threshold, such as an airflow rate less than or equal to 30 Lpm. The no inhalation event may occur when a subject does not inhale from the mouthpiece after opening the mouthpiece cover and during the measurement cycle. The no inhalation event may also occur when the subject's inspiratory effort is insufficient to ensure proper delivery of the medication via a flow pathway (e.g., the flow pathway 119), such as when the inspiratory effort generates insufficient airflow to activate a deagglomerator (e.g., such as the deagglomerator 121) and, thus, aerosolize the medication in a dosing cup (e.g., the dosing cup 116).

The low inhalation event may be associated with pressure measurement readings and/or airflow metrics within a particular range, such as an airflow rate greater than 30 Lpm and less than or equal to 45 Lpm. The low inhalation event may occur when the subject inhales from the mouthpiece after opening the mouthpiece cover and the subject's inspiratory effort causes at least a partial dose of the medication to be delivered via the flow pathway. That is, the inhalation may be sufficient to activate the deagglomerator such that at least a portion of the medication is aerosolized from the dosing cup.

The good inhalation event may be associated with pressure measurement readings and/or airflow metrics above the low inhalation event, such as an airflow rate which is greater than 45 Lpm and less than or equal to 200 Lpm. The good inhalation event may occur when the subject inhales from the mouthpiece after opening the mouthpiece cover and the subject's inspiratory effort is sufficient to ensure proper delivery of the medication via the flow pathway, such as when the inspiratory effort generates sufficient airflow to activate the deagglomerator and aerosolize a full dose of medication in the dosing cup.

The excessive inhalation event may be associated with pressure measurement readings and/or airflow metrics above the good inhalation event, such as an airflow rate above 200 Lpm. The excessive inhalation event may occur when the subject's inspiratory effort exceeds the normal operational parameters of the inhalation device 501a, 501b. The excessive inhalation event may also occur if the inhalation device 501a, 501b is not properly positioned or held during use, even if the subject's inspiratory effort is within a normal range. For example, the computed airflow rate may exceed 200 Lpm if the air vent is blocked or obstructed (e.g., by a finger or thumb) while the subject is inhaling from the mouthpiece.

Any suitable thresholds or ranges may be used to categorize a particular event. Some or all of the events may be used. For example, the no inhalation event may be associated with an airflow rate which is less than or equal to 45 Lpm and the good inhalation event may be associated with an airflow rate which is greater than 45 Lpm and less than or equal to 200 Lpm. As such, the low inhalation event may not be used at all in some cases.

The pressure measurement readings and/or the computed airflow metrics may also be indicative of the direction of flow through the flow pathway of the inhalation device 501a, 501b. For example, if the pressure measurement readings reflect a negative change in pressure, the readings may be indicative of air flowing out of the mouthpiece via the flow pathway. If the pressure measurement readings reflect a positive change in pressure, the readings may be indicative of air flowing into the mouthpiece via the flow pathway. Accordingly, the pressure measurement readings and/or airflow metrics may be used to determine whether a subject is exhaling into the mouthpiece, which may signal that the subject is not using the inhalation device 501a, 501b properly.

The inhalation device 501a, 501b may include a spirometer or similarly operating device to enable measurement of lung function metrics. For example, the inhalation device 501a, 501b may perform measurements to obtain metrics related to a subject's lung capacity. The spirometer or similarly operating device may measure the volume of air inhaled and/or exhaled by the subject. The spirometer or similarly operating device may use pressure transducers, ultrasound, or a water gauge to detect the changes in the volume of air inhaled and/or exhaled.

The data collected from, or calculated based on, the usage of the inhalation device 501a, 501b (e.g., pressure metrics, airflow metrics, lung function metrics, dose confirmation information, etc.) may be computed and/or assessed via external devices as well (e.g., partially or entirely). More specifically, a wireless communication circuit (e.g., such as the wireless communication circuit 129) in the electronics module may include a transmitter and/or receiver (e.g., a transceiver), as well as additional circuitry.

For example, the wireless communication circuit may include a Bluetooth chip set (e.g., a Bluetooth Low Energy chip set), a ZigBee chipset, a Thread chipset, etc. As such, the electronics module may wirelessly provide the data, such as pressure measurements, airflow metrics, lung function metrics, dose confirmation information, and/or other conditions related to usage of the inhalation device 501a, 501b, to a mobile device 502a, 502b, 502c. The electronics module may also send the timestamps associated with the data. The data may be provided in real time to the external device to enable exacerbation risk prediction based on real-time data from the inhalation device 501a, 501b that indicates time of use, how the inhalation device 501a, 501b is being used, and personalized data about the subject, such as real-time data related to the subject's lung function and/or medical treatment. The external device may include software for processing the received information and for providing compliance and adherence feedback to the subject via a graphical user interface (GUI), such as via a mobile device or via a computer associated with a HCP.

The airflow metrics may include personalized data that is collected from the inhalation device 501a, 501b in real-time, such as one or more of an average flow of an inhalation/exhalation, a peak flow of an inhalation/exhalation (e.g., a maximum inhalation received), a volume of an inhalation/exhalation, a time to peak of an inhalation/exhalation, and/or the duration of an inhalation/exhalation. The airflow metrics may also be indicative of the direction of flow through the flow pathway. That is, a negative change in pressure may correspond to an inhalation from the mouthpiece, while a positive change in pressure may correspond to an exhalation into the mouthpiece. When calculating the airflow metrics, the electronics module may be configured to eliminate or minimize any distortions caused by environmental conditions. For example, the electronics module may re-zero to account for changes in atmospheric pressure before or after calculating the airflow metrics. The one or more pressure measurements and/or airflow metrics may be timestamped and stored in the memory of the electronics module.

In addition to the airflow metrics, the inhalation device 501a, 501b, or another computing device, may use the airflow metrics to generate additional data. For example, the controller of the electronics module of the inhalation device 501a, 501b may translate the airflow metrics into other metrics that indicate the subject's lung function and/or lung health that are understood to medical practitioners, such as peak inspiratory flow metrics, peak expiratory flow metrics, and/or forced expiratory volume in 1 second (FEV1), for example. The electronics module of the inhalation device 501a, 501b may determine a measure of the subject's lung function and/or lung health using a mathematical model such as a regression model. The mathematical model may identify a correlation between the total volume of an inhalation and FEV1. The mathematical model may identify a correlation between peak inspiratory flow and FEV1. The mathematical model may identify a correlation between the total volume of an inhalation and peak expiratory flow. The mathematical model may identify a correlation between peak inspiratory flow and peak expiratory flow.

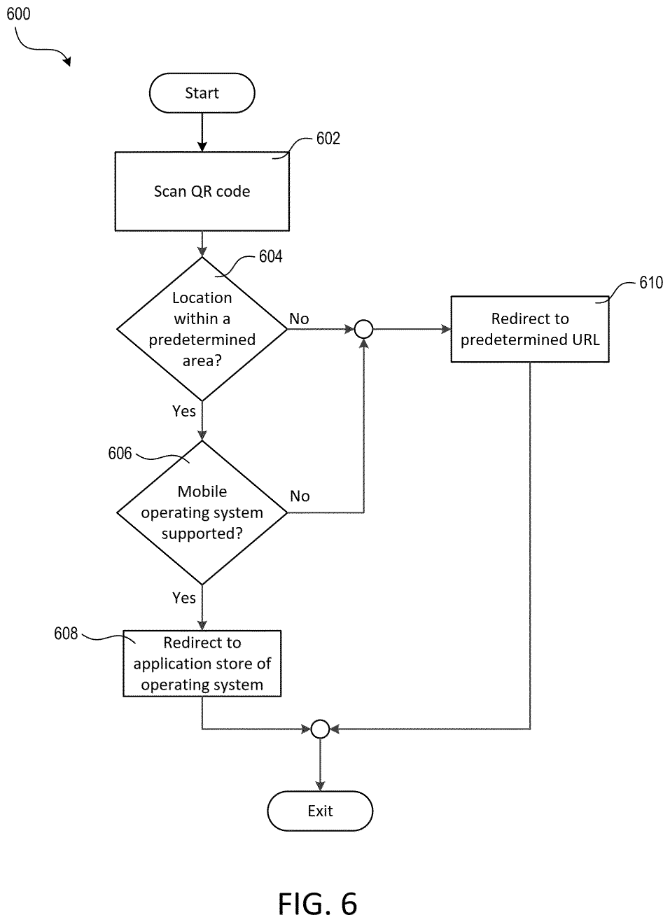

FIG. 6 is a flow diagram that illustrates an example procedure 600 for sending a Uniform Resource Locator (URL) request based on a location of an external device. Although described with reference to an external device (e.g., such as the mobile devices 502a, 502b, 502c shown in FIG. 5), other computing devices may perform at least a portion of the procedure 600. A user may scan the QR code on the inhalation device using the external device. When the user scans the QR code on the inhalation device, the external device may determine which URL to provide to the user. The external device may use the medicament type and/or a location associated with the inhalation device and/or the external device to determine which URL to provide to the user. For example, the URL(s) provided to the user may be associated with a predetermined generic URL, the medicament type, and/or location.

At 602, the external device may scan a QR code of an inhaler. The QR code may be located on a housing (e.g., such as the main housing 104 shown in FIGS. 1-3) of the inhaler. Additionally or alternatively, the QR code may be located on a cap (e.g., the top cap 102 shown in FIGS. 1-4) of the inhaler. For example, the QR code may be located on an interior or exterior surface of the cap. Additionally or alternatively, the QR code may be located on packaging associated with the inhaler. The external device may determine a URL and/or a medicament type of the inhaler based on the QR code. The URL may be associated with the medicament type. The QR code may indicate the medicament type and/or the URL. For example, the QR code may comprise an indication that indicates the type of medicament stored in the inhaler. The external device may determine a product identifier (ID) of the inhaler based on an image of the QR code. The external device may determine the medicament type based on the product ID. The product ID may comprise a multi-digit alphanumeric code that indicates the medicament type of the inhaler. The external device may be configured to determine a communication passkey that is unique to the inhaler based on the QR code. The external device may be configured to transmit the communication passkey to the electronics module of the inhaler to enable communication between the electronics module and the external device. The communication passkey comprises a Bluetooth Low Energy (BLE) passkey. The external device may scan, at 602, the QR code using the camera and/or camera application.

At 604, the external device may determine whether it is located (e.g., its location is) within a predetermined area (e.g., geographical area). For example, the external device may determine whether it is within a specific country (e.g., such as the United States) or a specific region (e.g., such as the European Union). The external device may be configured to determine its location (e.g., a location indication) based on a preconfigured setting of the external device. For example, the external device may use global positioning system (GPS) data, language setting data, and/or other settings data to determine its location. The external device may send an indication of its location to a server (e.g., that hosts the determined URL). In response to the location indication, the server may send an application store URL to the external device that is specific to the medicament type and/or the location of the external device.

When the external device's location is within the predetermined area, the external device may determine, at 606, whether a mobile operating system of the external device is supported by the determined URL (e.g., application store URL). For example, one or more operating systems (e.g., such as Android, IOS, etc.) may be supported by the determined URL.

When the external device's location is outside of the predetermined area, the external device may redirect, at 610, the user to a predetermined URL. The predetermined URL may comprise a homepage associated with the inhaler and/or medicament type or a support page associated with the inhaler and/or medicament type. If the external device's location is outside of the predetermined area, redirecting the external device to an application store URL not available in that area would result in an error. Instead of redirecting the external device to an error page, the external device outside of a predetermined area is redirected to a homepage associated with the inhaler and/or medicament type or a support page associated with the inhaler and/or medicament type.

When the external device's operating system is supported by the determined URL, the external device may redirect, at 608, to an application store associated with the operating system of the external device and/or the predetermined area (e.g., country). For example, the external device may send a request to the application store associated with the operating system of the external device (e.g., to download software that is specific to the inhaler and/or medicament type of the inhaler). The external device may receive software that is specific to the inhaler, the medicament type of the inhaler, and/or the location. The external device may install an application (e.g., mobile application) using the software that is specific to the inhaler, the medicament type of the inhaler, and/or the location.

When the external device's operating system is not supported by the determined URL, the external device may redirect, at 610, the user to a predetermined URL. The predetermined URL may comprise a homepage associated with the inhaler and/or medicament type or a support page associated with the inhaler and/or medicament type. If the external device's operating system is not supported by the determined URL, redirecting the external device to the determined URL would result in an error. Instead of redirecting the external device to an error page, the external device outside of a predetermined area is redirected to a homepage associated with the inhaler and/or medicament type or a support page associated with the inhaler and/or medicament type.

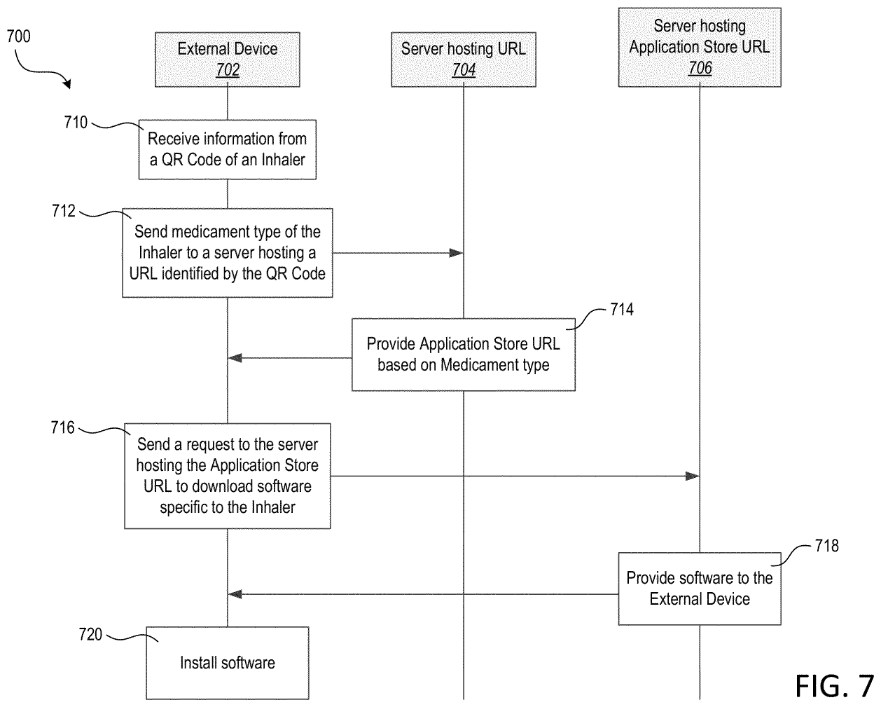

FIG. 7 is a flow diagram that illustrates an example procedure 700 for identifying a URL to visit based on a Quick Response (QR) code on an inhalation device (e.g., such as the inhalation device 100 shown in FIGS. 1-3). Although described with reference to an external device 702 (e.g., such as the mobile devices 502a, 502b, 502c shown in FIG. 5), a server 704 hosting a URL (e.g., such as the DHP 510 shown in FIG. 5), and a server 706 hosting an application store URL, other computing devices may perform at least a portion of the procedure 700. A user may scan the QR code on the inhalation device using the external device 702. When the user scans the QR code on the inhalation device, the external device 702 may determine which URL to provide to the user. The external device 702 may use the medicament type and/or a location associated with the inhalation device and/or the external device 702 to determine which URL to provide to the user. For example, the URL(s) provided to the user may be associated with the medicament type and/or location.

At 710, the external device 702 may receive information from a QR code of an inhaler. The QR code may be located on a housing (e.g., such as the main housing 104 shown in FIGS. 1-3) of the inhaler. Additionally or alternatively, the QR code may be located on a cap (e.g., the top cap 102 shown in FIGS. 1-4) of the inhaler. For example, the QR code may be located on an interior or exterior surface of the cap. Additionally or alternatively, the QR code may be located on packaging associated with the inhaler. The QR code may indicate a medicament type and/or a URL. For example, the QR code may comprise an indication that indicates the type of medicament stored in the inhaler. The external device 702 may receive, at 710, the information using the camera and/or camera application.

At 712, the external device 702 may send an indication of the medicament type and a location indication to the server 704 hosting the URL. For example, the external device 702 may launch a browser application and access a URL redirection URL at the server 704.

At 714, the server 704 may provide an application store URL to the external device 702. Each of a plurality of medicament types may be associated with a respective mobile application hosted by an application store URL. For example, the server 704 may determine which application store URL to provide based on the medicament type and/or the location indication. The server 704 may send, at 714, the application store URL to the external device 702 that corresponds with the medicament type and/or the location indication. The two-step process for obtaining the application store URL, described above, facilitates greater flexibility of implementation. Although in principle the application store URL could be encoded directly in the QR code itself, this would exclude the possibility of modifying the application store URL after the QR has been applied to the inhaler (or its packaging). In contrast, with the approach of the present example, although the URL embedded in the QR code is “fixed” (in the sense that it must remain accessible and functional at the hosting server 704), the URL of the application store can change over time. This can be handled by updating a redirection table at the server 704 to point to a new application store URL. Whenever an external device 702 accesses the redirection URL hosted by the server 704, the server will provide to that external device 702 the appropriate, currently-valid application store URL.

At 716, the external device 716 may send a request to the application store URL, for example, to download software that is specific to the inhaler and/or the medicament type of the inhaler. The software may comprise a mobile application that is configured to operate on the operating system of the external device 702.

At 720, the external device 702 may be configured to install the software (e.g., mobile application). The external device 702 may be configured to operate the software to provide information associated with the inhaler and/or medicament type of the inhaler. For example, the software may provide inhaler instructions, tutorial(s), usage feedback, medicament interactions, potential side effects, etc.

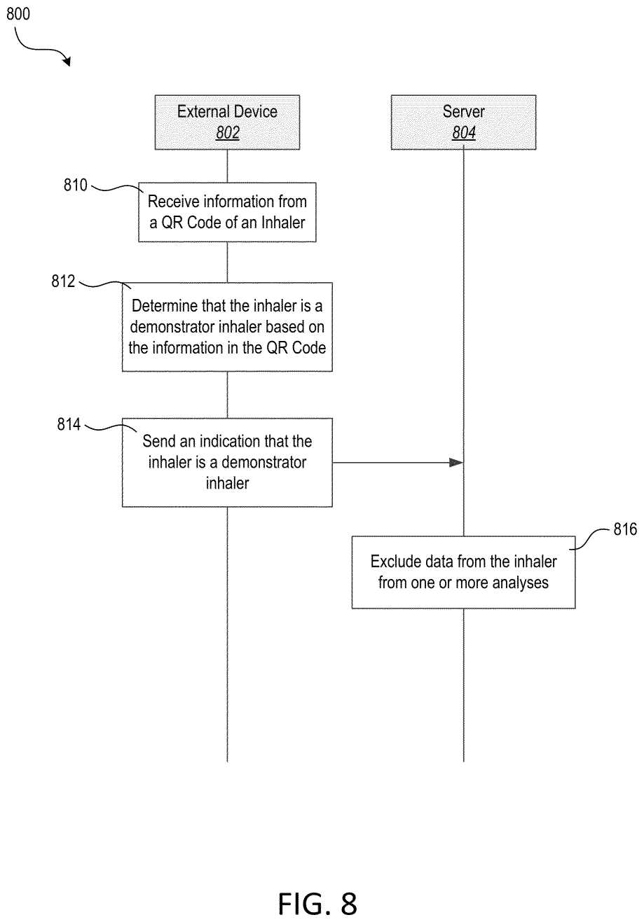

FIG. 8 is a flow diagram that illustrates an example procedure 800 for excluding data from a demonstrator inhaler from one or more analyses. Although described with reference to a server 804 (e.g., such as the DHP 510 shown in FIG. 5) and an external device 802 (e.g., such as the mobile devices 502a, 502b, 502c shown in FIG. 5), other computing devices may perform at least a portion of the procedure 800. It may be desirable to be able to determine whether one or more event records were generated by a demonstrator inhaler or an inhaler with medicament. The DHP may use the procedure 800 to determine whether an inhaler is a demonstrator inhaler or an inhaler with medicament. The DHP may determine to exclude data (e.g., event record(s)) from one or more analyses when the data has been received from a demonstrator inhaler.

At 810, the external device 802 may receive information from a QR code of an inhaler. The QR code may be located on a housing (e.g., such as the main housing 104 shown in FIGS. 1-3) of the inhaler. Additionally or alternatively, the QR code may be located on a cap (e.g., the top cap 102 shown in FIGS. 1-4) of the inhaler. For example, the QR code may be located on an interior or exterior surface of the cap. Additionally or alternatively, the QR code may be located on packaging associated with the inhaler. The QR code may indicate whether the inhaler is a demonstrator inhaler or an inhaler that comprises medicament. For example, the QR code may comprise an indication that the inhaler does not comprise medicament when the inhaler is a demonstrator inhaler.

At 812, the external device 802 may determine that the inhaler is a demonstrator inhaler based on the information in the QR code. For example, the external device 802 may analyze the information from the QR code to identify a demonstrator inhaler indication. The demonstrator inhaler indication may indicate that the inhaler is a demonstrator inhaler.

At 814, the external device 802 may send an indication (e.g., such as the demonstrator inhaler indication) to the server 804 that indicates that the inhaler is a demonstrator inhaler. For example, the external device 802 may send, at 814, inhaler data (e.g., one or more event records) associated with the inhaler to the server 804. The external device 802 may include the indication that the inhaler is a demonstrator inhaler in each of the one or more event records. The one or more event records may include a location associated with an inhaler event, one or more inhalation parameters, one or more environmental conditions, one or more usage parameters, and/or an actuation of an internal switch of the inhaler.

At 816, the server 804 may exclude data from the inhaler from one or more analyses. For example, the one or more analyses may include a predictive analysis associated with exacerbation. For example, the DHP may not use, at 816, the inhaler data received in the one or more event records in the one or more analyses when the inhaler is a demonstrator inhaler. The one or more analyses may include a predictive analysis associated with determining a probability of an asthma exacerbation in a user. Examples of predictive analyses for determining a probability of an asthma exacerbation in a user are described in greater detail in commonly-assigned U.S. Patent Publication No. US 2021/0106776A1, published on Apr. 15, 2021, entitled INHALER SYSTEM, the entire disclosure of which is hereby incorporated by reference.

It should be appreciated that identifying and excluding data generated by a demonstrator inhaler may improve the integrity of predictive analyses performed using inhaler data. If data generated by demonstrator inhalers were included in predictive analyses, less accurate analysis results would be calculated. The presence of data generated by a demonstrator inhaler may skew the ranges of data values used in the analyses. For example, baseline or assumed “normal” values calculated based on data generated by a demonstrator inhaler may be different from those that would be observed with a real (non-demonstrator) inhaler. This may lead to underestimation or overestimation of the probability of an exacerbation in the user's lung disease.

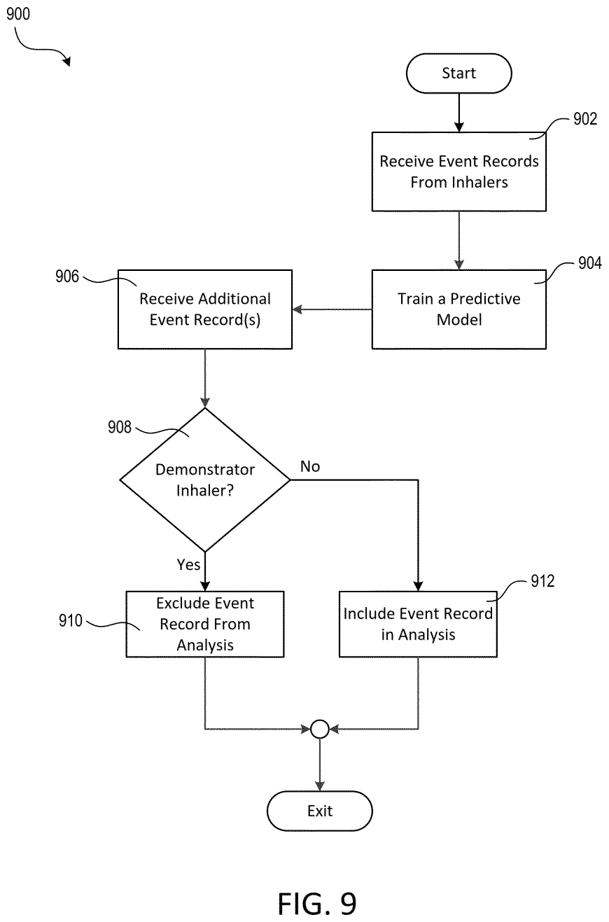

FIG. 9 is a flow diagram that illustrates an example procedure 900 for training a model to determine whether an inhaler is a demonstrator inhaler. Although described with reference to a digital health platform (DHP) (e.g., such as the DHP 510 shown in FIG. 5), other computing devices and/or software applications may perform the procedure 900. Some demonstrator inhalers may not include a demonstrator inhaler indication in their event records. For example, some demonstrator inhalers may not have a QR code. And, demonstrator inhalers may be identical to inhalers with medicament except the demonstrator inhalers have no medicament inside. In certain cases, an inhaler may be used to deliver a medicament at some times, and may be used as a demonstrator at other times. Thus, it may be important to be able to determine whether one or more event records were generated by a demonstrator inhaler or an inhaler with medicament. The DHP may use the procedure 800 to identify one or more characteristics that are associated with demonstrator inhalers. For example, the DHP may use the characteristic(s) associated with demonstrator inhalers to train a predictive model it can use to identify whether data (e.g., one or more event records) have been received from a demonstrator inhaler or an inhaler with medicament. The DHP may determine whether to include the data (e.g., event record(s)) in one or more analyses based on whether the data has been received from a demonstrator inhaler or an inhaler with medicament.

At 902, the DHP may receive a plurality of event records generated by a plurality of inhalers. The DHP may receive event records generated by demonstrator inhalers and inhalers with medicament. For example, the DHP may receive, at 902, a plurality of first event records generated by inhalers with medicament and a plurality of second event records generated by demonstrator inhalers. Each of the event records may be associated with a single inhaler (e.g., demonstrator inhaler or inhaler with medicament). Each of the event records may be associated with (and/or may include an indication of) a day and a time that the respective event record was generated. For example, the day and time may correspond with an inhaler event associated with the respective event record. Each of the event records may be associated with (and/or may include an indication of) a respective user of the single inhaler. Each of the event records may be associated with (and/or may include an indication of) a flow rate. The event records may be generated by the inhalers and sent to respective mobile devices (e.g., such as the mobile devices 502a, 502b, 502c shown in FIG. 5) that are paired with the inhalers. The mobile devices may then send the event records to the DHP. The event records may include an indication identifying whether the inhaler is a demonstrator inhaler. The DHP may receive the event records over an extended period of time (e.g., days, weeks, months).

The plurality of event records may be received from a plurality of known demonstration inhalers associated with demonstration programs and a plurality of known inhalers with medicament associated with clinical programs. For example, the dataset used for training the predictive model may include data from 247 known demonstrator inhalers and 477 known inhalers with medicament.

At 904, the DHP may train a predictive model using the plurality of event records. The predictive model may be trained, at 904, using an unsupervised learning method. The unsupervised learning method may comprise a clustering method, such as a k-means or c-means clustering method, for example. Alternatively, the predictive model may be trained, at 904, using a supervised learning method. The supervised learning method may include gradient boosted decision trees and/or an XGBoost algorithm. The XGBoost machine learning algorithm may be used for supervised binary classification, with sensitivity and specificity levels of approximately 0.91 and 0.93, respectively. The predictive model may identify one or more characteristics of an event record that is associated with demonstrator inhalers. For example, one or more of the following may be associated with demonstrator inhalers: inhaler events not occurring between a daily time period associated with nighttime, inhaler events not occurring on Saturday or Sunday, a user associated with the inhaler having an age that is above a lower threshold and/or below an upper threshold, a number of inhaler events that exceeds a predefined threshold (e.g., such as 200) within a predetermined time period, a dose count that exceeds an expected number of doses of medicament, and/or a percentage of flow rates of the event records associated with the inhaler being above a threshold flow rate. Appropriate weights may be assigned to each of the characteristics, at 904, when training the predictive model.

For example, a user may be less likely to use their demonstrator inhaler at night. Instead, an event record from a time period associated with nighttime is more likely to be from an inhaler with medicament. An inhaler event at night may signal that the user initiated the inhaler event because they needed a dose of medicament. That is, users may be less likely to wake up at night to practice inhalation using a demonstrator inhaler. Similarly, a user may be less likely to use their demonstrator inhaler on weekend days. Instead, an event record from a weekend day is more likely to be from an inhaler with medicament. An inhaler event during the weekend may signal that the user initiated the inhaler event because the needed a dose of medicament. That is, users may be less likely to practice inhalation using a demonstrator inhaler during a busy weekend. Young users and old users may be unlikely to have and/or use a demonstrator inhaler. A user may use a demonstrator inhaler frequently within a predetermined time period, for example, to become familiar with inhaling through the inhaler. Users may be less likely to frequently inhale through an inhaler with medicament, for example, because successive doses of medicament may be associated with adverse effects.

A user may be more likely to exceed a certain dose count when using a demonstrator inhaler. An inhaler with medicament may be pre-loaded with a certain number of doses of medicament. When event records exceeding the number of pre-loaded doses of medicament are received by the DHP, the inhaler may be considered to be a demonstrator inhaler. A user may be more likely to inhale through a demonstrator inhaler with irregular flow rates. For example, the user may attempt various inhalation flow rates through a demonstrator inhaler to become comfortable with the appropriate flow rates.

At 906, the DHP may receive one or more event records generated by an inhaler. Each of the one or more event records may be associated with a single inhaler (e.g., demonstrator inhaler or inhaler with medicament). Each of the event records may indicate a day and/or a time that the respective event record was generated. For example, the day and time may correspond with an inhaler event associated with the respective event record. Each of the event records may be associated with a respective user of the single inhaler. Each of the event records may indicate a flow rate associated with the respective inhaler event.

At 908, the DHP may determine whether the inhaler is a demonstrator inhaler (e.g., or an inhaler with medicament) using the trained predictive model. The DHP may analyze, at 908, the inhaler data to determine whether the inhaler is a demonstrator inhaler. The inhaler data may include one or more metadata included in the event record, one or more inhalation parameters, one or more environmental conditions, one or more usage parameters, and/or an actuation of an internal switch of the inhaler. The metadata may include an age of a user associated with the inhaler. The one or more inhalation parameters may include flow rate(s) associated with the one or more event records. The one or more environmental conditions may include temperature, humidity, etc. The one or more usage parameters may include a number of inhaler events, a dose count, etc. The DHP may assign weights to the inhaler data using the trained predictive model to determine whether the inhaler is a demonstrator inhaler.

When the DHP determines that the inhaler is a demonstrator inhaler, the DHP may exclude, at 910, the one or more event records from the inhaler from one or more analyses. For example, the one or more analyses may include a predictive analysis associated with exacerbation. For example, the DHP may not use, at 910, the inhaler data received in the one or more event records in the one or more analyses when the inhaler is not a demonstrator inhaler.

When the DHP determines that the inhaler is not a demonstrator inhaler (e.g., is an inhaler with medicament), the DHP may include, at 912, the one or more event records in one or more analyses (e.g., such as a predictive analysis associated with exacerbation). For example, the DHP may use, at 912, the inhaler data received in the one or more event records in the one or more analyses when the inhaler is not a demonstrator inhaler.

It should be appreciated that identifying and excluding data generated by a demonstrator inhaler may improve the integrity of predictive analyses performed using inhaler data. If data generated by demonstrator inhalers were included in predictive analyses, less accurate analysis results would be calculated.

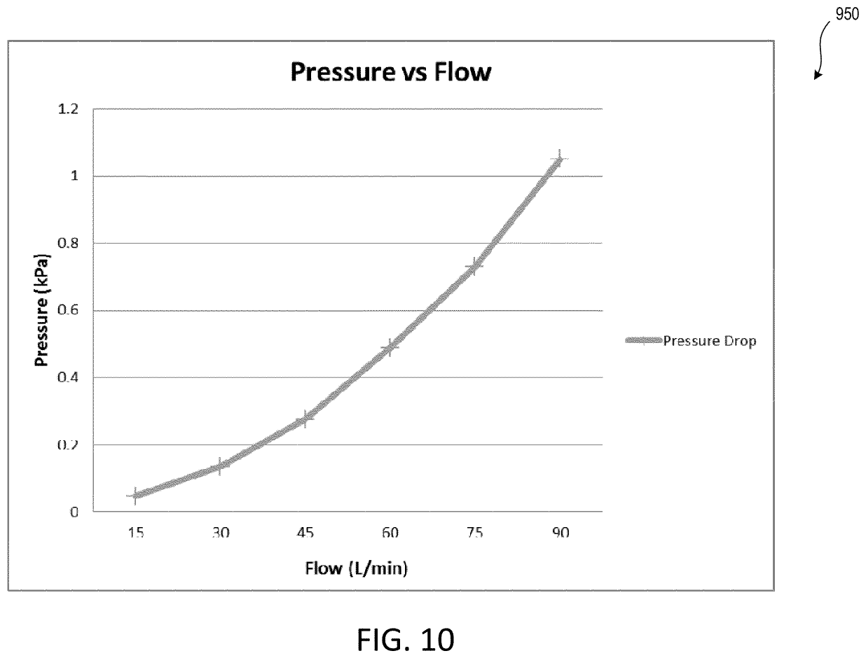

FIG. 10 is a graph 950 of exemplary airflow rates through an inhalation device (e.g., such as the inhalation device 100 shown in FIGS. 1-3 and/or the inhalation devices 501a, 501b shown in FIG. 5) based on pressure measurements calculated by a sensor system of an electronics module (e.g., such as the electronics module 120 shown in FIGS. 2-4) of the inhalation device. It will be appreciated that the graph 950 of airflow rates and pressure drops shown in FIG. 10 are merely examples, and may vary based on the size, shape, and design of the inhalation device and its internal components.

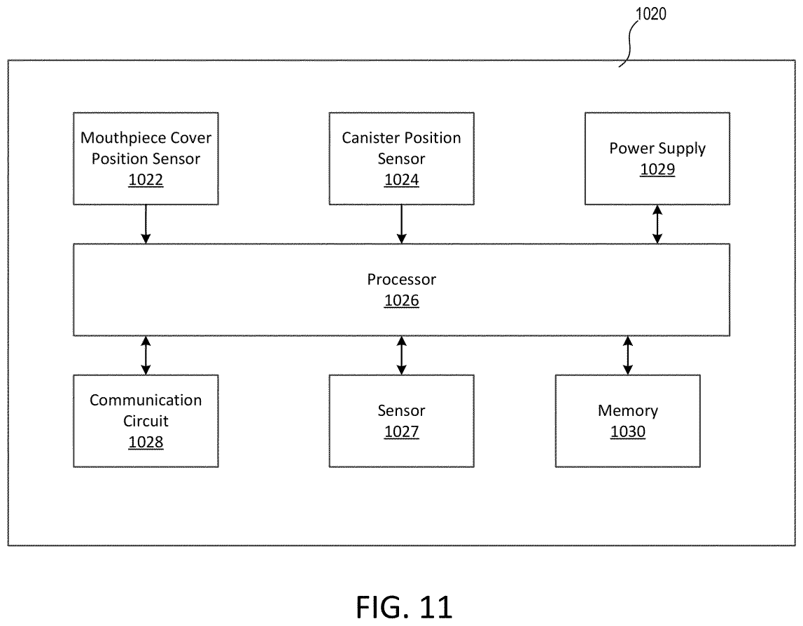

FIG. 11 is a block diagram of an example electronics module 1020 of an inhalation device (e.g., such as the inhalation device 100 shown in FIGS. 1-3 and/or the inhalation devices 501a, 501b shown in FIG. 5). The electronics module 1020 may be an example of the electronics module 120 shown in FIGS. 2-4. The electronics module 1020 may include a control circuit 1026 (e.g., a processor), a sensor system 1027, a communication circuit 1028, and a power supply 1029, such as a battery (e.g., such as the battery 126 shown in FIG. 4).

The processor 1026 may access information from, and store data in memory 1030 of the electronics module 1020. The memory 1030 may include any type of suitable memory, such as non-removable memory and/or removable memory. The non-removable memory may include random-access memory (RAM), read-only memory (ROM), a hard disk, or any other type of memory storage device. The removable memory may include a subscriber identity module (SIM) card, a memory stick, a secure digital (SD) memory card, and the like. The memory 1030 may be internal to the controller. The processor 1026 may also access data from, and store data in, memory that is not physically located within the electronics module 1020, such as on a server or a smartphone.

The memory 1030 may comprise a computer-readable storage media or machine-readable storage media that maintains computer-executable instructions for performing one or more as described herein. For example, the memory 1030 may comprise computer-executable instructions or machine-readable instructions that include one or more portions of the procedures described herein. The processor 1026 of the electronics module 1120 may access the instructions from memory for being executed to cause the processor 1026 of the electronics module 1120 to operate as described herein, or to operate one or more other devices as described herein. The memory 1030 may comprise computer-executable instructions for executing configuration software. For example, the computer-executable instructions may be executed to perform, in part and/or in their entirety, one or more procedures, such as the procedures 600, 700, 800, and/or 900 as described herein. Further, the memory 1030 may have stored thereon one or more settings and/or control parameters associated with the electronics module 1120.

The processor 1026 may include a microcontroller, a programmable logic device (PLD), a microprocessor, an application specific integrated circuit (ASIC), a field-programmable gate array (FPGA), and/or any suitable processing device or control circuit. The memory may include computer-executable instructions that, when executed by the processor 1026, cause the processor 1026 to implement the processes of the electronics module 1020 as described herein. When used herein, the terms controller and processor 1026 may be used interchangeably.

The sensor system 1027 may include one or more sensors, such as one or more pressure sensors, temperature sensors, humidity sensors, acoustic sensors, optical sensors, orientation sensors, and/or the like. The pressure sensor(s) may include a barometric pressure sensor (e.g., an atmospheric pressure sensor), a differential pressure sensor, an absolute pressure sensor, and/or the like. The sensors may employ microelectromechanical systems (MEMS) and/or nanoelectromechanical systems (NEMS) technology. The pressure sensor(s) may be configured to provide an instantaneous pressure reading to the processor 1026 of the electronics module 1020 and/or aggregated pressure readings over time. The pressure sensor(s) may be configured to measure a plurality of atmospheric pressures within the inhalation device. Examples of the sensors 1027 are described in reference to US 2020/0360630 A1, the entire disclosure of which are incorporated herein by reference. Further, it should be appreciated that the processor 1026 of the electronics module 1020 may be configured to convert pressure measurements received from the pressure sensor to a flow rate based on Bernoulli's principle (e.g., the Bernoulli/Venturi effect).

The electronics module 1020 (e.g., and/or a mobile application residing on an external device) may use measurements from the sensor system 1027 to determine one or more dosing events. For example, the electronics module 1020 may be configured to compare one or more measurements from the sensor system 1027 to one or more threshold values to categorize an inhalation event as a no/low inhalation event, a fair inhalation event, a good inhalation event, an excessive inhalation event, and/or an exhalation event. For example, the electronics module may generate a good inhalation event when the measurements from the sensor system 1027 indicate a flow rate in a particular range (e.g., greater than 20 L/min, or between 200 liters per min (L/min) and 20 L/min), generate a no inhalation event when the measurements from the sensor system 1027 indicate a flow rate that is less than a threshold value (e.g., 20 L/min), and an excessive inhalation event when the measurements from the sensor system 1027 indicate a flow rate that is greater than an upper threshold (e.g., greater than 200 L/min). Further, although described primarily in terms of flow rate, the measurements calculated by the sensor system may be used to calculate inhalation volume and/or inhalation duration, and the thresholds may be inhalation volume thresholds and/or inhalation duration thresholds.

The communication circuit 1028 may include a transmitter and/or receiver (e.g., a transceiver), as well as additional circuitry (e.g., such as a controller and/or memory). The communication circuit 1028 may include a wireless communication circuit. For example, the communication circuit 1028 may include a Bluetooth chip set (e.g., a Bluetooth Low Energy chip set), a ZigBee chipset, a Thread chipset, etc. As such, the electronics module 1020 may be configured to wirelessly provide data (e.g., the parameters determined by the processor 1026, such as pressure measurements, temperature, humidity level, orientation, etc., one or more recorded events, etc.) to an external device, including a smartphone. The external device may include software for processing the received information and for providing compliance and adherence feedback and/or any of the notifications described herein to users of the inhalation device via a graphical user interface (GUI).

The power supply 1029 may provide power to the components of the electronics module 1020. The power supply 1029 may be any suitable source for powering the electronics module 1020, such as a coin cell battery, for example. The power supply 1029 may be rechargeable or non-rechargeable. The power supply 1029 may be secured to the electronics module 1020 such that the power supply 1029 maintains continuous contact with and/or is in electrical connection with the components of a PCB of the electronics module 1020. The power supply 1029 may have a battery capacity that may affect the life of the power supply 1029. As will be further discussed below, the distribution of power from the power supply 1029 to the one or more components of the electronics module 1020 may be managed to ensure the power supply 1029 can power the electronics module 1020 over the useful life of the inhalation device 100 and/or the medication contained therein.

The electronics module 1020 may have a plurality of power states, each with respective power consumption levels. For example, the electronics module 1020 may be configured to operate in a system off state, a sleep state, a low power active state, and/or an active state. Each of the power states may be defined by different power consumption levels. For example, the electronics module 1020 may be configured to operate in a system off state, a sleep state, a low power active state, and/or an active state. The electronics module 1020 consumes the least amount of power while in the off state (e.g., no power or just enough to run a clock and/or monitor one or more processor pins in electrical communication with one or more contact pads), consumes more power in the sleep state than the off state (e.g., to drive the memory, the communication circuit, and/or a timer or clock), and consumes more power in the low power active state than in the sleep or off states, and consumes more power in the active state than in the low power active, sleep, or off states (e.g., to drive the processor 1026, the sensor system 1027, the communication circuit 1028, potentially in a faster advertising mode than the sleep state, and/or a timer or clock). Examples of the power states of an inhalation device, such as the inhalation device, are described in US 2018/0140786 A1, the entire disclosure of which is incorporated herein by reference.

While the electronics module 1020 is in the active state, the electronics module 1020 may operate in one or more modes, such as a measurement mode, a data storage/data processing mode, an advertising mode, and/or a connected mode. It should be appreciated that the electronics module 1020 may operate in multiple modes at one time (e.g., the modes may overlap).

In the measurement mode, the processor 1026 of the electronics module 1020 may power on the sensor system 1027. The processor 1026 may cause the sensor system 1027 to take pressure measurement readings, temperature readings, humidity readings, orientation readings, etc. for a predetermined time period (e.g., up to 60 seconds) and/or until a mouthpiece cover of the inhalation device (e.g., such as the mouthpiece cover 108 shown in FIGS. 1-3) is closed or no changes in pressure are detected. The processor 1026 may turn off one or more components of the electronics module 1020 while the sensor system 1027 is capturing readings to further conserve power. The sensor system 1027 may sample the readings at any suitable rate. For example, the sensor system 1027 may have a sample rate of 100 Hz and thus a cycle time of 10 milliseconds. The sensor system 1027 may generate a measurement complete interrupt after the measurement cycle is complete. The interrupt may wake the processor 1026 or cause it to turn on one or more components of the electronics module 1020. For example, after or while the sensor system 1027 is sampling one or more pressure measurements, temperature readings, humidity readings, orientation readings, etc., the processor 1026 may process and/or store the data and, if measurements are complete, power off the sensor system 1027.

In the data storage/data processing mode, the processor 1026 may power on at least a portion of the memory within the electronics module 1020. The processor 1026 may process the readings from the sensor system 1027 to compute, estimate, calculate or otherwise determine parameters (e.g., usage and/or storage conditions) and store the parameters in memory. The processor 1026 may also compare the readings and/or parameters to one or more thresholds or ranges to assess how the inhalation device is being used and/or the conditions under which the inhalation device is being used. Depending on the results of the comparison, the processor 1026 may drive one or more indicators to provide feedback to the user of the inhalation device. As noted above, the electronics module 1020 may operate in the measurement mode and the data storage/data processing mode simultaneously. After determining one or more parameters (e.g., usage and/or storage conditions) from the readings of the sensor system 1027, the processor 1026 may transmit the parameters and/or associated timestamps (e.g., based on the internal counter) to the external device when in the connected mode.

In the connected mode, the communication circuit 1028 may be powered on and the electronics module 1020 may be “paired” with an external device, such as a smartphone. The processor 1026 may retrieve data from the memory and wirelessly transmit the data to the external device. The processor 1026 may retrieve and transmit all of the data currently stored in the memory. The processor 1026 may also retrieve and transmit a portion of the data currently stored in the memory. For example, the processor 1026 may be able to determine which portions have already been transmitted to the external device and then transmit the portion(s) that have not been previously transmitted. Alternatively, the external device may request specific data from the processor 1026, such as any data that has been collected by the electronics module 1020 after a particular time or after the last transmission to the external device. The processor 1026 may retrieve the specific data, if any, from the memory and transmit the specific data to the external device.

Further, when connected with the external device, the electronics module 1020 may be configured to transmit Bluetooth special interest group (SIG) characteristics for managing access to data stored in the module 1020. The Bluetooth SIG characteristics may include one or more of a manufacturer name of the inhalation device, a serial number of the inhalation device, a hardware revision number of the inhalation device, and/or a software revision number of the inhalation device. When connected with the external device, the electronics module 1020 may retrieve data from memory and transmit the data to the external device.

The electronics module 1020 may include a mouthpiece cover position sensor 1022 and a canister position sensor 1024. The mouthpiece cover position sensor 1022 may be configured to sense the position of the mouthpiece cover (e.g., open or closed), and the canister position sensor 1024 may be configured to sense the position of a medication canister (e.g., such as the medication reservoir 110 shown in FIG. 2), for example, along a longitudinal axis within a main housing (e.g., such as the main housing 104 shown in FIGS. 1-3) such as in a first, second, or third position. In some examples, the mouthpiece cover position sensor 1022 may be a contact pad, and the canister position sensor 1024 may include one or more contact pads. As such, the processor 1026 may be configured to determine the position of the mouthpiece cover and/or determine when the position of the mouthpiece cover changes based on feedback from the mouthpiece cover position sensor 1022. Further, the processor 1026 may be configured to determine the position of the medication canister and/or determine when the position of the medication canister changes based on feedback from the canister position sensor 1024. The processor 1026 may be configured to timestamp and/or transmit data indicating the position of the mouthpiece cover and/or the medication canister to the external device.

The signals generated by a switch contact contacting the contact pads and/or the measurement readings taken by the sensory system 1027 may be timestamped and stored in memory of the electronics module 1020. The foregoing parameters may be indicative of various usage and/or storage conditions associated with the inhalation device. For example, as movement of a movable inner housing causes the switch contact to contact one or more of the contact pads, the processor 1026 may use the signals from the contact pads to record and timestamp each transition. Further, since the signals from the contact pads may correlate to the position of the mouthpiece cover (e.g., open or closed), the processor 1026 may be able to detect and track the position of the mouthpiece cover and/or medicament canister over time. It will be appreciated that the processor 1026 may be able to sense and track the status of the mouthpiece cover without interfering with the delivery of medication through the flow pathway of the inhalation device.

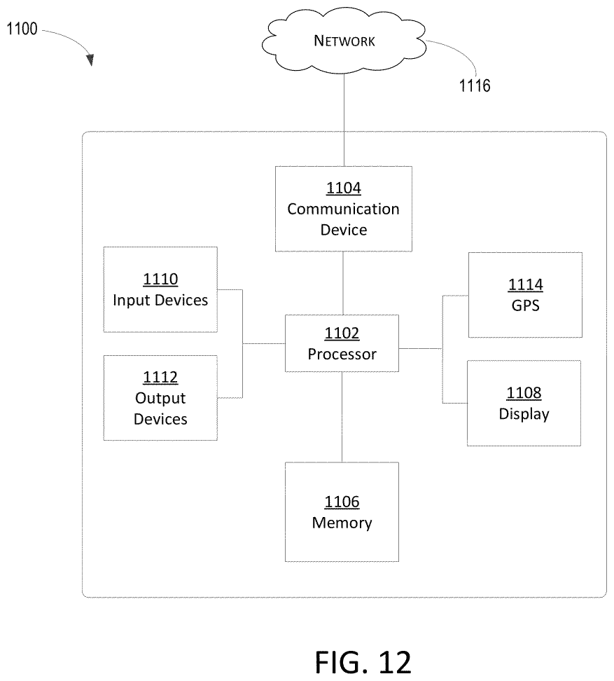

FIG. 12 illustrates a block diagram of an example computing device 1100 (e.g., external device and/or mobile device such as the mobile devices 502a, 502b, 502c shown in FIG. 5). The computing device 1100 may include a personal computer, such as a laptop or desktop computer, a tablet device, a cellular phone or smartphone, a server, or another type of computing device. The computing device 1100 may include a processor 1102, a communication interface 1104, a memory 1106, a display 1108, input devices 1110, output devices 1112, and/or a GPS circuit 1114. The computing device 1100 may include additional, different, or fewer components.

The processor 1102 may include one or more general purpose processors, special purpose processors, conventional processors, digital signal processors (DSPs), microprocessors, integrated circuits, a programmable logic device (PLD), application specific integrated circuits (ASICs), or the like. The processor 1102 may perform signal coding, data processing, image processing, power control, input/output processing, and/or any other functionality that enables the computing device 1100 to perform as described herein.

The processor 1102 may store information in and/or retrieve information from the memory 1106. The memory 1106 may include a non-removable memory and/or a removable memory. The non-removable memory may include random-access memory (RAM), read-only memory (ROM), a hard disk, or any other type of non-removable memory storage. The removable memory may include a subscriber identity module (SIM) card, a memory stick, a memory card, or any other type of removable memory. The memory may be local memory or remote memory external to the computing device 1100. The memory 1106 may store instructions which are executable by the processor 1102. Different information may be stored in different locations in the memory 1106.

The memory 1106 may comprise a computer-readable storage media or machine-readable storage media that maintains computer-executable instructions for performing one or more as described herein. For example, the memory 1106 may comprise computer-executable instructions or machine-readable instructions that include one or more portions of the procedures described herein. The processor 1102 of the external device 1100 may access the instructions from memory for being executed to cause the processor 1102 of the external device 1100 to operate as described herein, or to operate one or more other devices as described herein. The memory 1106 may comprise computer-executable instructions for executing configuration software. For example, the computer-executable instructions may be executed to perform, in part and/or in their entirety, one or more procedures, such as the procedures 600, 700, 800, and/or 900 as described herein. Further, the memory 1106 may have stored thereon one or more settings and/or control parameters associated with the external device 1100.

The processor 1102 that may communicate with other devices via the communication device 1104. The communication device 1104 may transmit and/or receive information over the network 1116, which may include one or more other computing devices. The communication device 1104 may perform wireless and/or wired communications. The communication device 1104 may include a receiver, transmitter, transceiver, or other device capable of performing wireless communications via an antenna. The communication device 1104 may be capable of communicating via one or more protocols, such as a cellular communication protocol, a Wi-Fi communication protocol, Bluetooth®, a near field communication (NFC) protocol, an internet protocol, another proprietary protocol, or any other radio frequency (RF) or communications protocol. The computing device 1100 may include one or more communication devices 1104.

The processor 1102 may be in communication with a display 1108 for providing information to a user. The information may be provided via a user interface on the display 1108. The information may be provided as an image generated on the display 1108. The display 1108 and the processor 1102 may be in two-way communication, as the display 1108 may include a touch-screen device capable of receiving information from a user and providing such information to the processor 1102. The processor 1102 may be configured to generate, on the display 1108, an indication of any event and/or dose record generated by and communication from the inhalation device to the external device 1100.

The processor 1102 may be in communication with a GPS circuit 1114 for receiving geospatial information. The processor 1102 may be capable of determining the GPS coordinates of the wireless communication device 1100 based on the geospatial information received from the GPS circuit 1114. The geospatial information may be communicated to one or more other communication devices to identify the location of the computing device 1100.

The processor 1102 may be in communication with input devices 1110 and/or output devices 1112. The input devices 1110 may include a camera, a microphone, a keyboard or other buttons or keys, and/or other types of input devices for sending information to the processor 1102. The display 1108 may be a type of input device, as the display 1108 may include touch-screen sensor capable of sending information to the processor 1102. The output devices 1112 may include speakers, indicator lights, or other output devices capable of receiving signals from the processor 1102 and providing output from the computing device 1100. The display 1108 may be a type of output device, as the display 1108 may provide images or other visual display of information received from the processor 1102.

The following embodiments are disclosed:
1. A system comprising:
an inhaler comprising medicament, an electronics module, and a Quick Response (QR) code, wherein the electronics module comprises a processor, memory, and a communication circuit; and
a computer-readable storage medium comprising executable instructions that, when executed by a processor of an external device, cause the processor of the external device to:
determine a Uniform Resource Locator (URL) based on the QR code;
determine a medicament type of the inhaler based on the QR code;
send an indication of the medicament type and a location indication to a server that hosts the URL, and in response, receive an application store URL that is specific to the medicament type; and
send a request to the application store URL to download software that is specific to the inhaler and the medicament type of the inhaler.
2. The system of embodiment 1, wherein the application store URL is specific to the country that the external device is located.
3. The system of embodiment 1, wherein the computer-readable storage medium comprises executable instructions that, when executed by the processor of the external device, cause the processor of the external device to:
determine a location of the external device; and
send an indication of the location of the external device to the server that hosts the URL, and in response, receive the application store URL that is specific to the medicament type and the location of the external device.
4. The system of embodiment 1, wherein the computer-readable storage medium comprises executable instructions that, when executed by the processor of the external device, cause the processor of the external device to:
determine a product identifier (ID) of the inhaler based on the image of the QR code; and
determine the medicament type based on the product ID.
5. The system of embodiment 4, wherein the product ID comprises a multi-digit alphanumeric code that indicates the medicament type of the inhaler.
6 The system of embodiment 1, wherein the computer-readable storage medium comprises executable instructions that, when executed by the processor of the external device, cause the processor of the external device to:
determine a communication passkey that is unique to the inhaler based on the QR code; and
transmit the communication passkey to the electronics module of the inhaler to enable communication between the electronics module and the external device.
7. The system of embodiment 6, wherein the communication passkey comprises a Bluetooth Low Energy (BLE) passkey.
8. The system of embodiment 1, wherein the processor of the external device is configured to determine the location indication based on a preconfigured setting of the external device.
9. The system of embodiment 1, wherein the processor of the external device is configured to receive, in response to sending the indication of the medicament type and the location indication, a URL associated with inhaler support.
10. The system of embodiment 9, wherein the processor of the external device receives the URL associated with inhaler support when a location of the external device is not within a predetermined region.
11. The system of embodiment 1, wherein the computer-readable storage medium comprises executable instructions that, when executed by the processor of the external device, cause the processor of the external device to:
receive software that is specific to the inhaler and the medicament type of the inhaler; and
install an application using the software that is specific to the inhaler and the medicament type of the inhaler.
12. A system comprising:
an inhaler comprising medicament, an electronics module, and a Quick Response (QR) code, wherein the electronics module comprises a processor, memory, and a communication circuit;
a first computer-readable storage medium comprising executable instructions that, when executed by a processor of an external device, cause the processor of the external device to:
   determine a Uniform Resource Locator (URL) based on the QR code;
   determine a medicament type of the inhaler based on the QR code; and
   send an indication of the medicament type and a location indication to a server that hosts the URL; and
a second computer-readable storage medium comprising executable instructions that, when executed by a processor of the server, cause the processor of the server to:
   receive the indication of the medicament type and a location indication at the URL;
   determine that the medicament type is associated with a mobile application that is specific to the inhaler and the medicament type;
   determine whether the location indication matches a predetermined value; and
   redirect the external device to an application store URL that is specific to the inhaler and the medicament type based upon a determination that the location indication matches the predetermined value;
wherein the first computer-readable storage medium further comprises executable instructions that, when executed by the processor of an external device, cause the processor of the external device to:
receive the application store URL that is specific to the medicament type; and
send a request to the application store URL to download software that is specific to the inhaler and the medicament type of the inhaler.
13. A system comprising:
an inhaler comprising an electronics module and a Quick Response (QR) code, wherein the electronics module comprises a processor, memory, and a communication circuit;
a computer-readable storage medium comprising executable instructions that, when executed by a processor of an external device, cause the processor of the external device to: receive an image of the QR code;
   determine, based on the image of the QR code, that the inhaler is a demonstrator inhaler; and
   send, to a remote server, an indication that the inhaler is a demonstrator inhaler; and
a second computer-readable storage medium comprising executable instructions that, when executed by a processor of the remote server, cause the processor of the remote server to:
   receive inhaler data associated with the use of the inhaler and the indication that the inhaler is a demonstrator inhaler; and
   determine, based on the indication that the inhaler is a demonstrator inhaler, to not use the inhaler data associated with the use of the inhaler in an analysis associated with a user of the inhaler.
14. The system of embodiment 13, wherein a demonstrator inhaler is an inhaler that does not comprise medicament.
15. The system of embodiment 13, wherein the inhaler further comprises a sensor configured to detect the one or more inhalation parameters associated with a use of the inhaler by a user;
wherein the processor of the electronics module is configured to send the one or more inhalation parameters to the external device; and
wherein the computer-readable storage medium comprises executable instructions that, when executed by the processor of an external device, cause the processor of the external device to:
receive the one or more inhalation parameters from the inhaler;
send, to the remote server, the one or more inhalation parameters along with the indication that the inhaler is a demonstrator inhaler.
16. The system of embodiment 13, wherein the processor of the external device is configured to determine a location of the external device based on a preconfigured setting of the external device.
17. The system of embodiment 16, wherein the processor of the external device is configured to send an indication of the location to the remote server.
18. The system of embodiment 13, wherein the analysis associated with the user is an artificial intelligence model.
19. The system of embodiment 13, wherein the analysis associated with the user is a predictive analysis associated with exacerbation.
20. The system of embodiment 13, wherein the inhaler data comprises one or more of an event record, one or more inhalation parameters, one or more environmental conditions, one or more usage parameters, or actuation of an internal switch of the inhaler.
21. A system comprising:
a first inhaler comprising a first electronics module, a first sensor, and a first Quick Response (QR) code, wherein the first electronics module of the first inhaler comprises a first processor, a first memory, and a first communication circuit, and the first sensor of the first inhaler is configured to detect a first inhalation parameter associated with a use of the first inhaler by a first user;
a first computer-readable storage medium comprising executable instructions that, when executed by a second processor of a first external device, cause the second processor of the first external device to:
   receive an image of the QR code of the first inhaler;
   determine, based on the image of the QR code of the first inhaler, that the first inhaler is a demonstrator inhaler; and
   send, to a remote server, the first inhalation parameter and an indication that the first inhaler is a demonstrator inhaler;
a second inhaler comprising medicament, a second electronics module, a second sensor, and a second QR code, wherein the second electronics module of the second inhaler comprises a third processor, a second memory, and a third communication circuit, and wherein the second sensor of the second inhaler is configured to detect a second inhalation parameter associated with a use of the second inhaler by a second user;
a second computer-readable storage medium comprising executable instructions that, when executed by a fourth processor of a second external device, cause the fourth processor of the second external device to:
   receive an image of the QR code of the second inhaler;
   determine, based on the image of the QR code of the second inhaler, that the second inhaler comprises medicament and is not a demonstrator inhaler; and
   send, to the remote server, the second inhalation parameter; and
a third computer-readable storage medium comprising executable instructions that, when executed by a fifth processor of the remote server, cause the fifth processor of the remote server to:
receive the first inhalation parameter associated with the first inhaler and the indication that the first inhaler is a demonstrator inhaler;
receive the second inhalation parameter associated with the second inhaler; and
determine, based on the indication that the first inhaler is a demonstrator inhaler, to use the second inhalation parameter but not the first inhalation parameter in an artificial intelligence model.
22. The system of embodiment 21, wherein a demonstrator inhaler is an inhaler that does not comprise medicament.
23. An inhaler comprising:
a housing comprising a mouthpiece and a mouthpiece cover, wherein the mouthpiece cover is configured to cover the mouthpiece;
an electronics module, wherein the electronics module comprises a processor, memory, and a communication circuit; and
a Quick Response (QR) code, wherein the QR code comprises an indication that the inhaler does not comprise medicament.
24. The inhaler of embodiment 23, wherein the QR code comprises an indication of a Uniform Resource Locator (URL) associated with the inhaler.
25. The inhaler of embodiment 24, wherein the URL comprises an application store URL associated with an inhaler application.
26. The inhaler of embodiment 23, wherein the indication indicates that the inhaler is a demonstrator inhaler when the inhaler does not comprise medicament.
27. A method for training a predictive model that is configured to identify demonstrator inhalers that do not comprise medicament, the method comprising:
receiving a plurality of first event records generated by a plurality of first inhalers, wherein each of the plurality of first event records is associated with a single inhaler of the plurality of first inhalers, associated with a day and a time of a respective one of the plurality of first event records, associated with a respective user of a plurality of first users, and associated with a flow rate;
receiving a plurality of second event records generated by a plurality of second inhalers, wherein the plurality of second inhalers are demonstrator inhalers that do not comprise medicament, wherein each of the plurality of second event records is associated with a single inhaler of the plurality of second inhalers, a day and a time of a respective one of the plurality of second event records, a respective user of a plurality of second users, and a flow rate;
training a predictive model, using the plurality of first and second event records, to identify one or more parameters that are associated with demonstrator inhalers;
receiving a third event record generated by a third inhaler; and
determining, using the trained predictive model, whether the third inhaler is a demonstrator inhaler that does not comprise medicament.
28. The method of embodiment 27, wherein the predictive model is trained to determine that the third inhaler is a demonstrator inhaler based on inhalation events of the third inhaler not occurring between a daily time period associated with nighttime.
29. The method of embodiment 27, wherein the predictive model is trained to determine that the third inhaler is a demonstrator inhaler based on inhalation events of the third inhaler not occurring on Saturday or Sunday.
30. The method of embodiment 27, wherein the predictive model is trained to determine that the third inhaler is a demonstrator inhaler based on a user associated with the third inhaler having an age that is above a lower threshold or below an upper threshold.
31. The method of embodiment 27, wherein the predictive model is trained to determine that third inhaler is a demonstrator inhaler based on the third inhaler being associated with a number of inhalation events that exceeds a threshold (e.g., 200 uses) within a predetermined time period.
32. The method of embodiment 27, wherein the predictive model is trained to determine that the third inhaler is a demonstrator inhaler based on the third inhaler having a dose count that exceeds an expected number of doses of medicament (e.g., greater than 70 doses in an inhaler that traditionally only has 60 doses).
33. The method of embodiment 27, wherein the predictive model is trained to determine that the third inhaler is a demonstrator inhaler based on a percentage of the flow rates of a plurality of event records associated with the third inhaler being above a threshold flow rate.
34. The method of embodiment 27, wherein the predictive model is a first predictive model, the method further comprising:
training a second predictive model, using the plurality of first event records from the plurality of first inhalers; and
determining, using the second predictive model, a likelihood of a respiratory exacerbation of a respective user of the plurality of first users.
35. The method of embodiment 34, further comprising:
receiving a fourth inhaler record from a fourth inhaler; and
determining, using the second predictive model, a likelihood of a respiratory exacerbation of a user associated with the fourth inhaler.
36. The method of embodiment 27, wherein the predictive model is trained using an unsupervised learning method such as a k-means clustering method or a c-means clustering method.
37. The method of embodiment 27, wherein the predictive model is trained using a supervised learning method such as gradient boosted decision trees or an XGBoost algorithm.
38. The method of embodiment 27, wherein an event record is generated by an inhaler in response to an inhaler event.
39. The method of embodiment 38, wherein the inhaler event comprises actuation of a switch or receipt of measurements from a sensor.

What is claimed is:

1. A method for training a predictive model that is configured to identify demonstrator inhalers that do not comprise medicament, the method comprising:
receiving a plurality of first event records generated by a plurality of first inhalers, wherein each of the plurality of first event records is associated with a single inhaler of the plurality of first inhalers, associated with a day and a time of generation of the respective one of the plurality of first event records, associated with a respective user of a plurality of first users, and associated with a flow rate;
receiving a plurality of second event records generated by a plurality of second inhalers, wherein the plurality of second inhalers are demonstrator inhalers that do not comprise medicament, wherein each of the plurality of second event records is associated with a single inhaler of the plurality of second inhalers, and each includes an indication of any one or any two or more of: a day and a time of generation of the respective one of the plurality of second event records, a respective user of a plurality of second users, and a flow rate;
training a predictive model, using the plurality of first and second event records, to identify one or more parameters that are associated with demonstrator inhalers;
receiving one or more third event records generated by a third inhaler; and
determining, using the trained predictive model, and based on the one or more third event records, whether the third inhaler is a demonstrator inhaler that does not comprise medicament.

2. The method of claim 1, wherein the predictive model is trained to determine that the third inhaler is a demonstrator inhaler based at least in part on times associated with inhalation events of the third inhaler.

3. The method of claim 1, wherein the predictive model is trained to determine that the third inhaler is a demonstrator inhaler based at least in part on days associated with the inhalation events of the third inhaler.

4. The method of claim 1, wherein the predictive model is trained to determine that the third inhaler is a demonstrator inhaler based at least in part on an age of a user associated with the third inhaler.

5. The method of claim 1, wherein the predictive model is trained to determine that third inhaler is a demonstrator inhaler based at least in part on a number of inhalation events associated with the third inhaler.

6. The method of claim 1, wherein the predictive model is trained to determine that the third inhaler is a demonstrator inhaler based at least in part on a dose count associated with the third inhaler.

7. The method of claim 1, wherein the predictive model is trained to determine that the third inhaler is a demonstrator inhaler based at least in part on one or more flow rates of a plurality of event records associated with the third inhaler.

8. The method of claim 1, wherein the predictive model is a first predictive model, the method further comprising:
training a second predictive model, using the plurality of first event records from the plurality of first inhalers; and
determining, using the second predictive model, a likelihood of a respiratory exacerbation of a respective user of the plurality of first users.

9. The method of claim 8, further comprising:
receiving a fourth inhaler record from a fourth inhaler; and
determining, using the second predictive model, a likelihood of a respiratory exacerbation of a user associated with the fourth inhaler.

10. The method of claim 1, wherein the predictive model is trained using an unsupervised learning method.

11. The method of claim 1, wherein the predictive model is trained using a supervised learning method.

12. The method of claim 1, wherein an event record is generated by an inhaler in response to an inhaler event.

13. The method of claim 12, wherein the inhaler event comprises actuation of a switch or receipt of measurements from a sensor.

14.-39. (canceled)

40. The method of claim 2, wherein the predictive model is trained to determine that the third inhaler is a demonstrator inhaler based at least in part on the inhalation events not occurring during a daily time period associated with nighttime.

41. The method of claim 3, wherein the predictive model is trained to determine that the third inhaler is a demonstrator inhaler based at least in part on the inhalation events not occurring on Saturday or Sunday.

42. The method of claim 4, wherein the predictive model is trained to determine that the third inhaler is a demonstrator inhaler based at least in part on the user having an age that is above a lower threshold or below an upper threshold.

43. The method of claim 5, wherein the predictive model is trained to determine that third inhaler is a demonstrator inhaler based at least in part on the third inhaler being associated with a number of inhalation events that exceeds a threshold within a predetermined time period.

44. The method of claim 6, wherein the predictive model is trained to determine that the third inhaler is a demonstrator inhaler based at least in part on the third inhaler having a dose count that exceeds an expected number of doses of medicament.

45. The method of claim 7, wherein the predictive model is trained to determine that the third inhaler is a demonstrator inhaler based at least in part on a percentage of the flow rates of the plurality of event records associated with the third inhaler being above a threshold flow rate.

46. The method of claim 10, wherein the unsupervised learning method comprises a k-means clustering method or a c-means clustering method.
